(19) United States
(12) Patent Application Publication
(10) Pub. No.: US 2016/0122701 A1
(43) Pub. Date: May 5, 2016

(54) Methods, Devices and Systems for Algae Lysis and Content Extraction

(71) Applicant: CARNEGIE MELLON UNIVERSITY, Pittsburgh, PA (US)

(72) Inventors: C. Fred Higgs, III, Pittsburgh, PA (US); Philip R. LeDuc, Pittsburgh, PA (US); Kristin M. Warren, Pittsburgh, PA (US); Jeremiah N. Mpagazehe, Pittsburgh, PA (US)

(21) Appl. No.: 14/764,906

(22) PCT Filed: Jan. 31, 2014
(86) PCT No.: PCT/US14/13970

§ 371 Date: Jul. 30, 2015

Related U.S. Application Data
(60) Provisional application No. 61/849,712, filed on Feb. 1, 2013.

(51) Int. Cl. C12M 1/42; C12N 1/12; G06F 17/50; C12N 1/06

(52) U.S. Cl. CPC C12M 35/04; C12N 1/066; C12N 1/12; G06F 17/5009

(57) ABSTRACT

Described herein are devices, systems and methods for lysing algae cells, for production of a lysate product such as a biofuel. The systems and methods use a passive device that lyses the cells through flow configurations, geometries, and surfaces that would induce different stresses and negative pressure on the microalgae cells. When the stress is designed to exceed the mechanical strength of the microalgae cells, the cells are lysed, causing, e.g., lipid release which can be used to produce biofuels. Through an internally-created computational framework, the concept is validated and can be optimized for the lowest energy input with the highest level of lipid release. Also provided herein are computer-implemented methods for optimizing lysis in such systems and computer-readable media containing instructions for performing the computer-implemented methods.

### CROSS REFERENCE TO RELATED APPLICATIONS

This application claims the benefit of U.S. Provisional Patent Application No. 61/849,712, Filed Feb. 1, 2013, which is incorporated herein by reference in its entirety.

### STATEMENT REGARDING FEDERAL FUNDING

This invention was made with government support under National Science Foundation CMMI0645124 and the National Aeronautics and Space Administration NNXU9AK96H. The government has certain rights in this invention.

In the Energy Independence and Security Act of 2007, the United States government mandated the use of 36 billion gallons of renewable biofuels, annually, by the year 2022. To help meet this quota, biofuels, such as algae-based biodiesel, are gaining much attention because algae can be farmed on non-arable land, do not compete with food production, and have a high growth rate. Algae is a large group of photosynthetic, aquatic organisms that lack the roots, stems, and leaves of higher plants. Algae are extremely biodiverse and have the ability to accumulate lipids. Algae can be classified into two groups: macroalgae which are multicellular, and microalgae which are unicellular. Algae biomass is an attractive source of biofuel due to its high productivity and its ability to thrive in, otherwise, unusable areas. Producing biofuels from algae thus increases utilization of low-quality water sources and terrain without impeding upon nutrient-rich agricultural land.

The lipid extraction process is a crucial precursor step in which the oil is removed from the algae cells. Energy consumed during this process reduces the economic feasibility of algae-based biofuel as an alternative to conventional fossil fuels. Mechanical methods are particularly attractive for lipid extraction because they do not require harsh, expensive chemicals nor the production of intense microwaves. However, most devices that have been designed to perform mechanical lipid extraction, such as French press homogenizers, were designed with more consideration for preserving the integrity of the cell contents than for consideration of energy usage at industrial scales. Developing new mechanical technologies to actively lyse (rupture) algae cells in order to extract the lipids can be a costly trial-and-error process. An economical and efficient method to lyse algae cells is therefore very desirable.

### SUMMARY

To make microalgal biofuels an efficient energy source, microalgal biofuel processes must be optimized to reduce the energy consumption within the dewatering (separating the liquid and solid algal particulates), cell lysis (cell membrane rupture), and lipid acquisition stages. The systems and methods described herein facilitate lysis of algae for applications such as, but not limited to, biofuel production. The passive/semi-passive process lyses the cells through flow configurations, geometries, and surfaces that induce different stresses and negative pressure on the microalgae cells. When the stress induced through passive/semi-passive flow processes is exceeds the mechanical strength of the microalgae cells, the cells are lysed, causing lipid release. Through an internally-created computational framework, the concept is validated and can be optimized for the lowest energy input with the highest level of lipid release. As shown herein, in one embodiment of this device passive methods can be employed to lyse algae as they flow from a photobioreactor to a downstream lipid extraction processes in order to reduce the amount of energy needed to extract the lipids.

This invention invokes fluid dynamics and cell mechanics concepts to lyse cells passively that would decrease the energy consumption of the algal biofuel process. This passive system uses in-flow and surface interactions instead of energy-intensive processes, such as mechanical screw presses, French presses, etc., that are currently employed. These processes minimize use, or remove the requirement of chemicals that can alter the composition and effectiveness of the algae contents as well as increase the cost of the algae biofuel process. This stark difference in approach solves the inefficiencies of biofuel production helping to make it a viable candidate as a renewable energy source.

According to one embodiment of the methods described herein, a computational framework is used to predict the energy consumed during mechanical lipid extraction for algae-based biofuel production and/or shear stresses on algae cells. The method models fluid-induced shear stress applied to the cell, in combination with the motion and physical characteristics of individual algae cells, which are simulated by a combination of computational fluid dynamics (CFD) and discrete element method (DEM), respectively. The current work is unique in that it presents a low-energy configuration for algae lipid extraction. A computer-generated model is created to simulate the performance of this configuration.

By modeling the algae lipid extraction and the fluid flow around the algae cell, this work displays the relationship between algae cell lipid extraction efficiency and energy consumption. Understanding this relationship, and having models to predict it, are important in the development of a viable, low-energy lipid extraction processes for algae-based biofuel production. It was also found that the model can be used to predict the areas in the flow where lipid extraction is most likely to occur. Such information can be used to fabricate the novel lipid extraction device modeled in this work to minimize energy consumption during lipid extraction for algae-based biofuel production.

A system is therefore provided for producing an algae lysate from an algae culture in an algae culture medium. The system comprises a reservoir having a fluid outlet adapted for removal of fluid from the reservoir; a fluid conduit attached to the fluid outlet and comprising one or more lysing structures comprising a fluid flow path and a flow-altering structure that modifies the flow path to generate sufficient force to lyse an algae cell in the algae culture in at least a portion of the flow path at a flow rate generated by either a gravitational head of water of 40 meters or less, 60 psi or less or 30 W or less. In use, the system comprises algae in algae culture medium within the reservoir at a level above the fluid outlet of the reservoir. In one embodiment, the flow rate is less than or equal to a gravity flow rate. In another embodiment, the flow rate is produced by a pressure of 60 psi or less. In yet another embodiment, the flow rate is generated by a gravitational head of water of between 8 and 40 meters. In a further embodiment, the fluid flow, and the flow rate is produced by 48,000 or less Joules (J) per Liter of the algae culture in the algae culture medium, and in one embodiment between 8 J and 48,000 J per Liter of the algae culture in the algae culture medium. In one embodiment, the lysing structure lyses at least 5%, 10%, 25%, 50% or 75% of the cells at the flow rate.

The system comprises one or more lysing structures and in one embodiment, comprises two or more lysing structures. In one embodiment, the flow-altering structure is a protuberance within the flow path. In another, the flow-altering structure is a restriction in a diameter of the flow path, for example, a venturi. In yet another embodiment, the flow-altering structure is a bend in the flow path. In a further embodiment, the lysing structure at the flow rate produces turbulent flow in the lysing structure and walls of the flow path comprise a surface roughness (feature, texture, etc.) extending into the flow path beyond a turbulent flow viscous sublayer for the medium at the passive flow rate.

Also provided herein is a method of preparing a product from an algae lysate. The method comprises: growing microalgae in an aqueous medium in a photobioreactor; lysing the microalgae to produce a lysate by flowing the microalgae in the aqueous medium through the lysing structure according to any embodiment described herein; and producing a product from the lysate. According to one embodiment, the product is a biofuel that is prepared from a hydrophobic fraction of the lysate. In another embodiment, prior to lysing the microalgae, the microalgae is pretreated to reduce their bursting strength. According to certain embodiments, the microalgae are pretreated with either a raised temperature or lowering pH to reduce their bursting strength.

Also provided herein is a method of lysing algae cells, comprising flowing the microalgae in the aqueous medium through the lysing structure according to any embodiment described herein.

A computer-implemented method of optimizing algae lysis in a lysing structure through which algae is flowed in an aqueous medium also is provided. The method comprises: inputting aqueous medium physical data including temperature, viscosity, density, and solid-fraction, into a fluid dynamics modeling system within a flow domain that geometrically defines a flow path of the aqueous medium through the lysing structure; inputting algae physical data including size and bursting strength into a particle modeling system in the flow domain; calculating stresses on the algae in the flow domain for a given aqueous medium flow rate and/or energy input through the flow path; and determining a rate of lysis of cells for the flow rate and/or energy input through the flow path. In one embodiment, the method further comprises repeating at least the steps of calculating stresses on the algae in the flow domain for a given aqueous medium flow rate and/or energy input through the flow path; and determining a rate of lysis of cells for the flow rate and/or energy input through the flow path for a different flow rate and/or energy input and/or flow domain and comparing the results to optimize cell lysis percent and flow rate and/or energy input. A non-transitory computer-readable medium comprising instructions for performing the computer-implemented method in a processor also is provided.

### BRIEF DESCRIPTION OF THE DRAWINGS

FIG. 1 (a-f) depicts schematically non-limiting examples of flow-altering structures in lysing structures: (a) channel occlusion (b) network of channel occlusions (c) stagnation and recirculation (d) restriction orifice (e) surface texturing (f) obstacles in the flow.

FIG. 2 (A-C) depicts schematically non-limiting examples of the system for lysing algae described herein.

FIG. 3 is a schematic depiction of a computer.

FIG. 4 is a flow diagram describing an embodiments of a computer-implemented lysis optimization method.

FIG. 5 illustrates a Force/Distance curve as generated by an Atomic Force Microscope (AFM) where (1) is the approach of the cantilever tip, (2) is the cantilever snapping to the sample, (3) is the contact of the AFM tip and the sample, (4) is the adhesion between the sample and the tip, which usually overlaps (3), (5) is the pull off, and (6) is after the tip is removed from the sample the retraction of the tip.

FIG. 6 shows images of Scenedesmus dimorphus on AFM and the respective force/distance curves, where the green crosshairs is the place of measurement.

FIG. 7A shows a representative AFM 3D height image showing the surface topography of a dried S. dimorphus cell. FIG. 7B shows AFM topographical image of a typical single, dried S. dimorphus cell with points 1, 2, and 3 selected for nanoindentation to determine the Young's Modulus.

FIG. 8A shows a representative AFM force vs indentation approach curve from a defined nanoindentation location on single, dried S. dimorphus cell. In this experiment, the Young's modulus was calculated to be 26.85 MPa using a Hertzian contact model. FIG. 8B shows a histogram of binned Young's Modulus values from the designated locations on the same cell.

FIG. 9A shows a representative AFM force vs indentation approach curve for a S. dimorphus cell in solution. The Young's modulus here was calculated to be 3.95 MPa using a Hertzian contact model assumption. FIG. 9B shows a histogram of binned Young's Modulus values from the representative locations on the same cell.

FIG. 10 shows a comparison of dried and aqueous Young's modulus measurements of Scendesmus dimorphus cells. The error bars represent standard deviation with p≦0.05.

FIG. 11 shows 3D images of a single dried S. dimorphus cell.

FIG. 12 shows topography image from Asylum Research MFP-3D-AFM with, points selected for indentation. The cell shown in FIG. 12 is the same cell as shown in FIG. 11.

FIG. 13A shows Young's Modulus for the cell shown in FIG. 12. FIG. 13B provides Young's Modulus values for various substances.

FIG. 14 shows S. Dimorphus at 63× with Bodipy dye under bright field (FIG. 14A) and fluorescence (FIG. 14B) microscopy.

FIG. 15 shows OD results indicating that shearing is a good candidate for algal cell lysing.

FIG. 16 provides a shear stress contour plot and quantitative stress values for Example 3.

FIG. 17A shows a contour plot of the velocity field and FIG. 17B shows a contour plot of the pressure field for Example 3.

FIG. 18 is a model for calculating the forces on the particles after a collision using the spring-dashpot model.

FIG. 19 depicts a DEM particle, representing an algae cell, surrounded by a CFD element.

FIG. 20A displays the computational domain with the obstacle. In FIG. 20B, the computational mesh for the CFD is displayed. In FIG. 20A the fluid domain is shown as an outline around the obstacle placed in flow, and in FIG. 20B the fluid domain is shown as a wireframe to display CFD mesh.

FIG. 21, depicts the effect of varying cp. The occlusion ratios are set to 0.2 (FIG. 21A), 0.6 (FIG. 21B), or 0.8 (FIG. 21C).

Images of the supernatant streamlines and algae, as they flow over the obstacle, are displayed in FIG. 22, which shows supernatant streamlines and algae as they move around the obstacle after 60 ms of simulation time. In FIG. 22A, a trimetric view is displayed. In FIG. 22B, a side view is displayed

FIG. 23 provides images illustrating the motion of the algae as they move through the domain. FIG. 23 shows algae positions and supernatant velocity magnitude contour at different times during the simulation. Intact algae are displayed in gray, lysed algae are black. Shown are initial conditions (FIG. 23A), after 20 ms (FIG. 23 B), after 40 ms (FIG. 23C), after 60 ms FIG. 23D), and after 80 ms (FIG. 23E).

FIG. 24 depicts the cumulative percentage of lysed cells vs. time.

FIG. 25 displays the pressure drop across the length of the domain. This pressure is plotted along the white line in FIG. 25A. The figure shows contour of the pressure in the domain (FIG. 25A) and a graph of the pressure drop across the length of the domain (FIG. 25B).

FIG. 26 shows the energy consumed and the percentage of algae cells lysed.

FIG. 27 shows the contour of the strain-rate of the fluid in the domain (FIG. 27A) and a graph of the strain rate across the length of the domain (FIG. 27B).

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

Provided herein are devices and methods for lysing microalgae to obtain their cell contents, such as lipids for producing biofuels and precursors thereof. As indicated above, production of biofuels from microalgae needs to become commercially practicable before it becomes an acceptable energy alternative to fossil fuels. It is therefore desirable to lyse microalgae with little or no required energy input.

According to a first embodiment, lysing structures are provided. The lysing structures comprise a body and a fluid flow path within the body. The fluid flow path comprises one or more flow-altering structures capable of producing shear stresses able to lyse microalgae cells used in biofuels lipid production within fluid flowing through the flow path under low-energy flow.

Considering that it takes tens, if not thousands of liters of algae culture to generate the energy equivalent of one liter of gasoline 8-10 kWh, and that a single solar panel can produce in excess of a kWh under many circumstances, the sensitivity to energy costs of algal-based biofuels is abundantly illustrated, as is the need for a passive method of cell lysis. The devices, systems and methods described herein are as energy-neutral as possible, and are a significant step in achieving economic feasibility of an algal biofuels system. Further, economic efficiencies are further enhanced by the nature of the lysis structures in that there are no moving parts or requirements for additional chemical reagents.

In one embodiment, low-energy flow refers to gravity-feed conditions, that is at a pressure of no more than a water column of approximately 40 meters, or less than 60 psi, for example and without limitation in a range of from eight to 40 meters (26-132 feet or 11-57 psi). In the alternative, the low energy flow refers to a flow of liquid generated by 48,000 or less Joules (J) per Liter of the algae culture in the algae culture medium, and in one embodiment between 8 J and 48,000 J per Liter of the algae culture in the algae culture medium. According to another embodiment, the low energy flow refers to continuous flow rates achievable with 30 Watt (W) of power or less, for example from 5 mW to 30 W. The flow-altering structures of the fluid flow path produce laminar, turbulent, and/or cavitation flow patterns which impart sufficiently large stresses over the fluid path to rupture microalgae cells. It is not required that all cells are lysed, but that a sufficiently large portion of the cells are lysed. In one embodiment, the lysing structure lyses at least 5%, 10%, 25%, 50% or 75% of the cells at the flow rate. The remaining, unlysed cells can be discarded, recycled, recirculated, buried for carbon storage, or even lysed by active lysis methods using, for example, a mechanical screw or French press. As indicated herein, the overall goal is to reduce energy input into the lysing system and the passive lysis of a sufficiently large proportion of the cells before active lysis techniques would significantly impact production costs.

The low energy input requirements are particular to the lysis of microalgae in the production of biofuels on a commercial scale. Typical cell culturing and cell transfer processes seek to reduce cell stress and lysis. As an example, Born et al. (“Estimation of Disruption of Animal Cells by Laminar Shear Stress,” Biotechnology and Bioengineering 40:1004-1010 (1992)) illustrate stresses capable of disrupting mammalian hybridoma cells using a viscometer in the presence of dextran, a rheology modifier, that was required to achieve sufficient shear stresses. Born et al. sought to understand the limits of stress an animal cell, such as a hybridoma cell could withstand in order to prevent or minimize cell disruption in bioreactors, such as commercial bioreactors. In contrast, the methods, devices and systems seek to optimize cell lysis.

In cases where cell lysis is required for experimentation purposes, or typical larger-scale manufacturing processes, minimizing energy is atypical. As such, mechanical presses or other active methods with higher energy requirements can be utilized. Thus, the application of low-energy/low cost lysis methods is particular to the production of lipids for biofuels, and is not a typical consideration in cell culture and processing techniques—though the current device can be used in non-biofuel applications as well. An example of a low energy input would be roughly equal to or less than the energy to lift a quantity of water or algal culture medium sufficiently high to gravity-feed the described system. As an example, the amount of energy capable of lifting a liter of water 100 feet at sea level is approximately 83 mWh (milliwatt-hours). Taking that as an example, and allowing for pumping inefficiencies, to lift a liter of water 100 feet would require less than 200 mWh. Thus, considering 100 feet elevation to be excessive for the purposes herein, energy from a single 200 W solar panel can lift thousands of gallons a day to a sufficient elevation for purposes described herein. Thus “low energy” and “low energy requirements” refers to 30 Watt or less during continuos lysing or the equivalent of a pressure of no more than a water column of approximately 40 meters, or less than 60 psi, for example and without limitation in a range of from eight to 40 meters (26-132 feet or 11-57 psi). According to a preferred embodiment, the lysing structure is gravity-fed, for example by draining of a reservoir of algae-containing liquid under the force of gravity, such as from a drain located at a lower portion or bottom of the reservoir such that flow of the liquid through the lysing structure is achieved by gravity-feed and/or from pressure derived from a water column of the reservoir.

In the context of the lysis structures, systems and methods described herein, the shear forces to be applied to the algae cells are sufficient to rupture the cells. Different organisms and even species have different requirements for cell rupture, depending on a variety of factors including cell size and the presence of and integrity of a cell wall, the composition of which can affect the physical stresses required to rupture the cell. Table A provides examples of forces needed to rupture a variety of cell types. For reference, C. eugamentos is a microalgae.

TABLE A
Comparison of cell types concerning cell strength/bursting pressure

| | Cell Strength/ | |
|---|---|---|
| Cell Type | Bursting Pressure | Source |
| Mammalian cells | 2.4 (+/−) 0.21 uN | Zhang et al, 1991 (1) |
| S. typhimurim | 100 atm (10.13 MPa) | Carpita, 1985 (2) |
| (bacteria) | | |
| C. eugamentos (algae) | 95 atm (96.26 MPa) | Carpita, 1985 (2) |
| Yeast cells | 96 uN | Smith et al, 2000 (3) |
| V. terrestris (algae) | 455-532 kPa | Mine et al, 2006 (4) |
| Potato | 0.35 MPa (yield stress) | Waldron et al, 1995 (5) |

These values were obtained from different means. Sources 2 and 4 obtain the bursting pressure by pressurizing the cells and monitoring when they burst. Source 1 and 3 used a compression test between two optic fibers. Source 5 uses a tensile test specialized for these type of samples.
(1) Zhang, Z. et al., “A Novel Micromanipulation Technique for Measuring the Bursting Strength of Single Mammalian Cells,” Appl. Microbiol. Biotechnol. (1991) 36:208-210.
(2) Carpita, N.C., “Tensile Strength of Cell Walls of Living Cells, Plant Physiol. (October 1985) 79(2):485-488
(3) Smith et al., “Wall Material Properties of Yeast Cells: Part 1. Cell Measurements and Compression Experiments,” Chem. Eng. Sci. (2000) 55(11):2031-2041.
(4) Mine, I, et al. “Cell Wall Extensibility During Branch Formation in the Xanthophycean Alga Vaucheria terrestris,” Planta (2007) 226:971-979.
(5) Waldron, K W, et al., “New Approaches to Understanding and Controlling Cell Separation in Relation to Fruit and Vegetable Texture,” Trends in Food Science and Technology (1997) 8(7):213-221

The lysis structures described herein includes one or more features (flow-altering structures) capable of producing areas of turbulence and/or fluidic shear able to lyse a sufficient quantity of a particular cell, yet maintain a sufficient flow rate. For example, the lysis structure may rupture only 10% of the cells passing through, but a sample can be fed through the same structure multiple times or a series of structures such that the device number and overall percentage of cells ruptured increases.

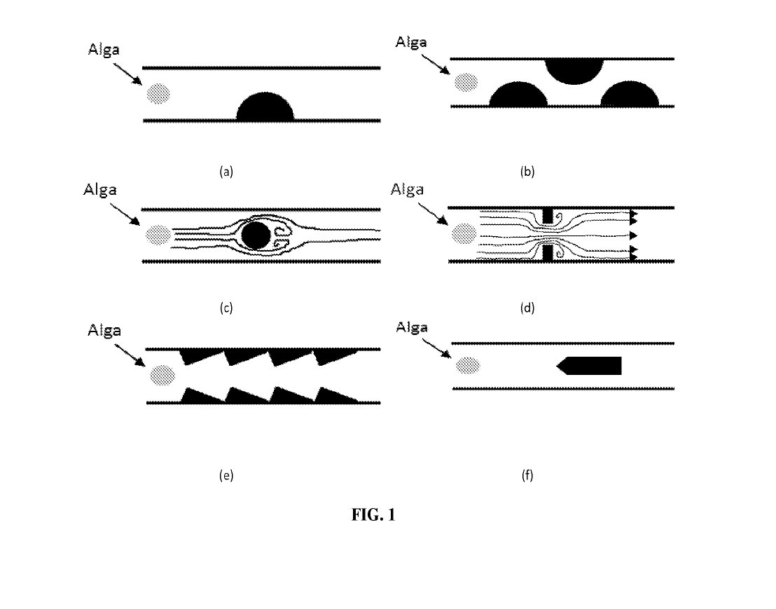

The body of the lysis structure may be a tube or pipe, but is a solid object with one or more flow paths comprising passages having an inlet and an outlet and a flow-altering structure therein. The passage(s) may have any geometric configuration. The body may comprise a pipe or tube having a substantially-circular cross section, as they are common plumbing supplies. Flow-altering structures include, without limitation occlusions or protuberances within the passage(s), affecting flow within the passage, bends in the passage or narrowings of the passage, which cause a restriction and/or divergent flow paths, or alterations in the wall of the fluid path that cause differentials in friction and therefore perturbations in laminar or turbulent flow, thereby producing, for example: turbulence; shear; laminar flow; rapid velocity (speed plus direction), directional or speed changes; pressure changes; and/or cavitation. Non-limiting examples of useful structures are depicted in FIG. 1. In one embodiment, the structure is a venturi, e.g., a narrowing or restriction of the fluid path followed by a subsequent expansion of the fluid path. In another embodiment, the structure comprises a surface roughness or surface features extending into the flow path. In one embodiment, the roughness disrupts a laminar or turbulent flow through the lysing structure. For example, in classical turbulent flow models, velocity profiles include four discrete layers extending from the wall: the viscous sublayer, the buffer layer, the overlap layer and the turbulent layer (See, e.g., Cengel, Y. A. and Cimbala, J. M., Fluid Mechanics: Fundamentals and Applications, 1^{st} ed., McGraw-Hill (2006), Chapter 8, “Flow in Pipes”). Therefore in one embodiment, the surface roughness extends at least beyond the turbulent flow viscous sublayer into the buffer layer in order to significantly affect flow profiles. The surface roughness may be in the form of grit or particles adhered to an inner surface of the lysing structure, ridges, etc., and the roughness may be combined with other flow-altering structures in the flow path. The result of the flow of liquids through the flow path and about the flow-altering structure(s) is localized stresses that are greater than the forces needed to rupture cells in liquid passing through the lysis structure at a flow rate that is achieved by low-energy and preferably passive means, such as by gravity flow.

A system for passively producing an algae lysate is therefore provided, comprising a reservoir having a fluid outlet fluidly connected to a lysing structure. In one embodiment, the reservoir is a photobioreactor in which algae are grown, and in another embodiment the reservoir is an independent vessel into which the algae culture is transferred once it is propagated. Water passes through the outlet and into the lysing structure at a low flow rate. In one embodiment, the flow rate is a gravity feed flow rate that is the fluid flow rate inherent to the system when fluid is passed through the outlet and lysing structure by force of gravity, either by downhill flow of the fluid, by siphoning, or by pressure generated by the column of fluid in the reservoir. In another embodiment, a pump is used to pump fluid from the reservoir and through the lysing structure preferably using a minimum of energy, with a maximum of from 5 mW to 30 W, or gravity feed of a column of water of 40M or less (less than 60 psi), or alternately the flow is produced by an energy input of 48,000 or less Joules (J) per Liter of the fluid (e.g., algae culture in the algae culture medium), and in one embodiment between 8 J and 48,000 J per Liter of the fluid. The lysing structure has one or more flow-altering structures able to produce a physical stress capable of rupturing a microalgae cell at the flow rate. The physical stress may be due to any stress produced in the flow of the fluid media and the surface texture, resulting from, for example and without limitation, shear, turbulence or cavitation.

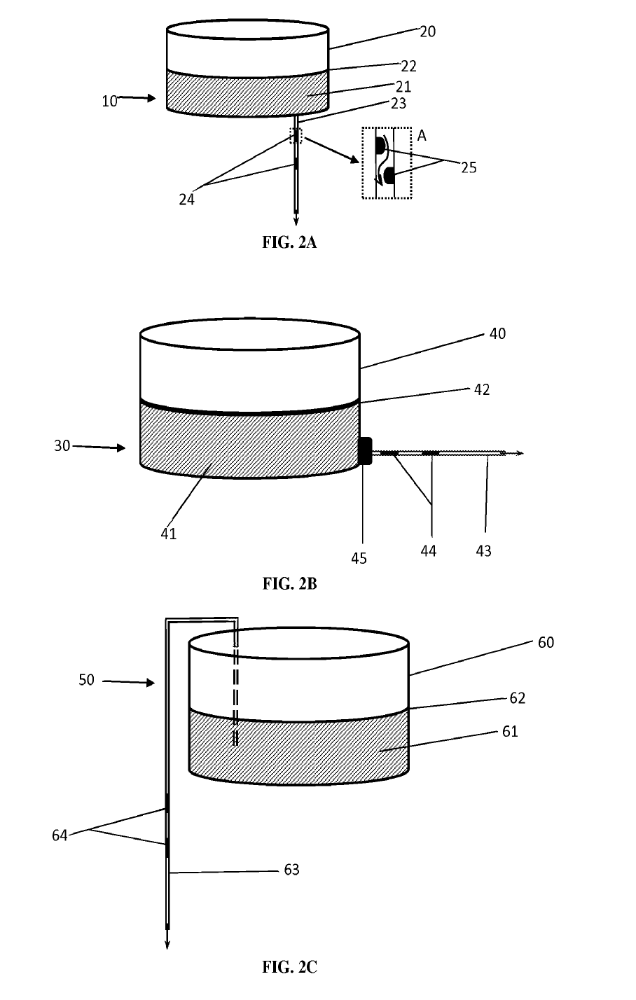

As would be understood by those of ordinary skill in the art, the lysing structure and flow-altering structure can have any suitable configuration so long as the structure, at the flow rate of the system, is able to lyse algae cells and, at times, extract the contents from the algae cells. FIGS. 2A, 2B and 2C show schematically alternate embodiments of a system described herein. Like reference numbers refer to similar structures. FIG. 2A shows a first schematic embodiment of system 10 for lysing algae cells. A reservoir 20 is shown containing algae-containing medium 21 filling the reservoir 20 to the fill line 22. Outlet 23 is shown having lysing structures 24. In this figure, details of the upper lysing structure 24 is shown in an expanded cross-sectional view A in which flow-altering structures 25 are depicted. Algae-containing medium passes through outlet 23, optionally controlled by a valve (not shown), such as a solenoid, and passed through the lysing structures 24, as shown by the arrows. The curved arrow in view A shows redirection of the flow of liquid through the lysing structures 24 by the flow-altering structures. This configuration produces significant pressures at the outlet and across the lysing structures due to the water column due to the height of the liquid in the reservoir. In this embodiment, at some point water would have to be raised to its final height in the reservoir.

FIG. 2B shows schematically a second variant of the system 30, with a reservoir 40, algae-containing medium 41 and a fill-line 42, but with outlet 43 being fluidly connected to reservoir 40 just below the fill line. Fluid is pumped out of the reservoir 40 by pump 45 and through the lysing structures 44. Of note, this requires energy to power the pump 45. As described herein, the power required by the pump ranges from 5 mW to 30 W, the energy requirements are 48,000 or less Joules (J) per Liter of the algae culture in the algae culture medium, and in one embodiment between 8 J and 48,000 J per Liter of the algae culture in the algae culture medium, or the pump generates less than 60 psi).

FIG. 2C shows schematically a second variant of the system 50, with a reservoir 60, algae-containing medium 61 and a fill-line 62, but with outlet 63 being fluidly connected to reservoir 60 as a siphon. Fluid is drawn out of the reservoir and is pulled through the lysing structures 64. There is a maximum height for the siphon. As above, a valve (not shown) may be employed to control liquid flow through the lysing structures.

As indicated above, FIGS. 2A-2C are schematic in nature and designs may be optimized for any reason. Multiple outlets and one to hundreds, or more, lysing structures may be employed in series or in parallel to most effectively lyse cells.

The configuration of the lysing structure is preferably optimized based on maximizing lysis at low flow rates. A computer-implemented example of such a process is provided below. The low flow rate is constrained by the maximum passively-achievable velocity—that is a velocity achievable without the assistance of a pump, such as by gravity-fed water flowing through a passage, for example under pressure of a water column of a reservoir of algae-containing medium. A maximum, passively-achievable velocity is used as a constraint in one embodiment because use of a pump requires energy input and adds an undesirable level of complexity to the system. Flowing the algae through an outlet containing the lysing structure where the outlet drains or siphons below an upper surface of liquid in the reservoir would obviate any requirement for actively pumping the liquid through the lysing structure.

By “medium” or “media,” it is meant an aqueous composition capable of sustaining growth of algae, and is typically and primarily salt water or fresh water, typically supplemented by suitable nutrients.

According to one embodiment, a method of preparing a microalgae lysate is provided. The method comprises passively flowing a fluid comprising microalgae through a lysis structure as described herein. The fluid is flowed through the lysis structure at or below a maximum passively-achievable velocity.

In a variation of the method, the cells are pre-treated prior to flowing the fluid through the lysing structure in order to lower the required bursting force. Many pre-treatments are expected to cost-effectively modify the algae cells so that lower forces are needed to burst the cells (Kunzek et al. “Aspects of material science in food processing: changes in plant cell walls of fruits and vegetables, Z Lebensm Unters Forsch A (1999) 208:233-250). For example, the cells may be heated prior to lysing to reduce their bursting strength (Hülsheger, H., et al. “Killing of Bacteria with Electric Pulses of High Field Strength,” Radiat Environ Biophys (1981) 20:53-65 and Waugh, R, et al., “Thermoelasticity of Red Blood Cell Membrane,” Biophys. J. Volume 26 Apr. 1979 115-132). Heating may be simply accomplished in a solar system, such as with a photobioreactor. Significant temperature rises can be accomplished simply by running the solution containing the cells through a typical solar water heating system akin to those used for home heating. Also, changing the pH of the solution can weaken the cells, thereby reducing their bursting strength (Valent et al. The Structure of Plant Cell Walls: V. On The Binding of Xyloglucan To Cellulose Fibers, Plant Physiol. (1974) 54, 105-108). Many acids, such as sulfuric acid, are inexpensive and can be added to the cells at any point prior to lysis, for example in a reservoir, such as a reservoir shown in FIGS. 2A-2C that is not the primary photobioreactor. Other chemical or enzymatic treatments may be employed to assist in the lysis of the algae

In another embodiment, a method of preparing a biofuel comprising preparing a microalgae lysate, as described above and subsequently preparing a biofuel product from the lysate by extracting lipids from the lysate by any useful method, and subsequently converting the lipids to a biofuel. In one non-limiting embodiment, algae debris and water is separated from lipids (e.g., triglycerides) by settling or centrifugation. Triglycerides are hydrolyzed in a base, such as sodium hydroxide (lye) to produce fatty acids. Esterification of the fatty acids produce biodiesel, which can be accomplished by base-catalyzed transesterification with an alcohol such as methanol or ethanol, or by acid-catalyzed esterification with an alcohol. Triglycerides can be mixed with appropriate amounts of a base and an alcohol at the same time to produce biodiesel—fatty acid alkyl esters. A large variety of methods are known and understood by those of ordinary skill to extract and clarify oils from cell lysates, and producing a biofuel, such as biodiesel from the oils.

In another embodiment, a computer-implemented method is provided for modeling lysis of algae within a lysis structure as described above. The method includes modeling an interaction between a fluid representing medium in which algae is lysed and algae cells within the physical constraints of a flow path in a lysing structure. Modeling such an interaction is performed by a modeling system comprising computer instructions implemented using any effective programming language, such as C++, and a processor. The computer instructions include instructions for modeling fluid dynamics of the algae media, instructions for modeling the algae, within the constraints/geometries of a specified flow path in a lysing structure.

As used herein a “modeling system” is a computational framework by which an algae lysis and extraction structure or system is modeled. A modeling system embodies various elements of the algae lysis structure or system. As used herein an “element,” in the context of modeling a system, means any components of the model, as described above, including, without limitation, fluid dynamic parameters, discrete element parameters, global data, initialization process data, data layers, representative grids and images, and input parameters. For example, elements of the C++ platform, described below include, for example: fluid dynamics parameters, discrete element parameters, lysing structure geometry, etc.

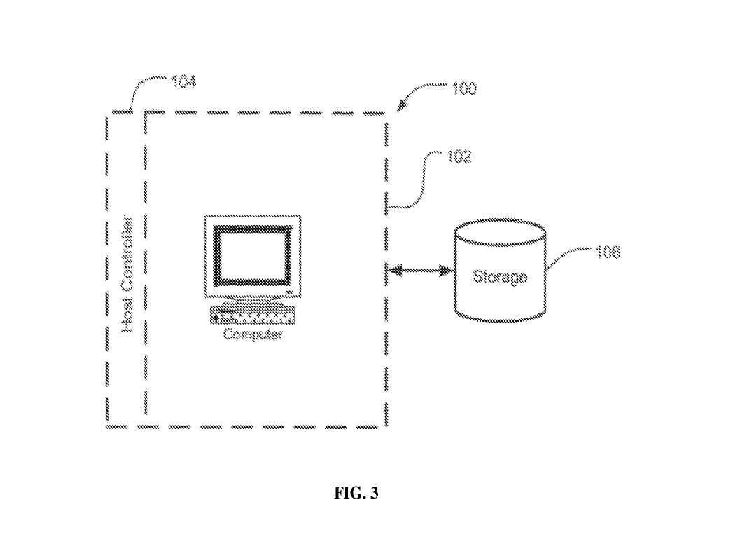

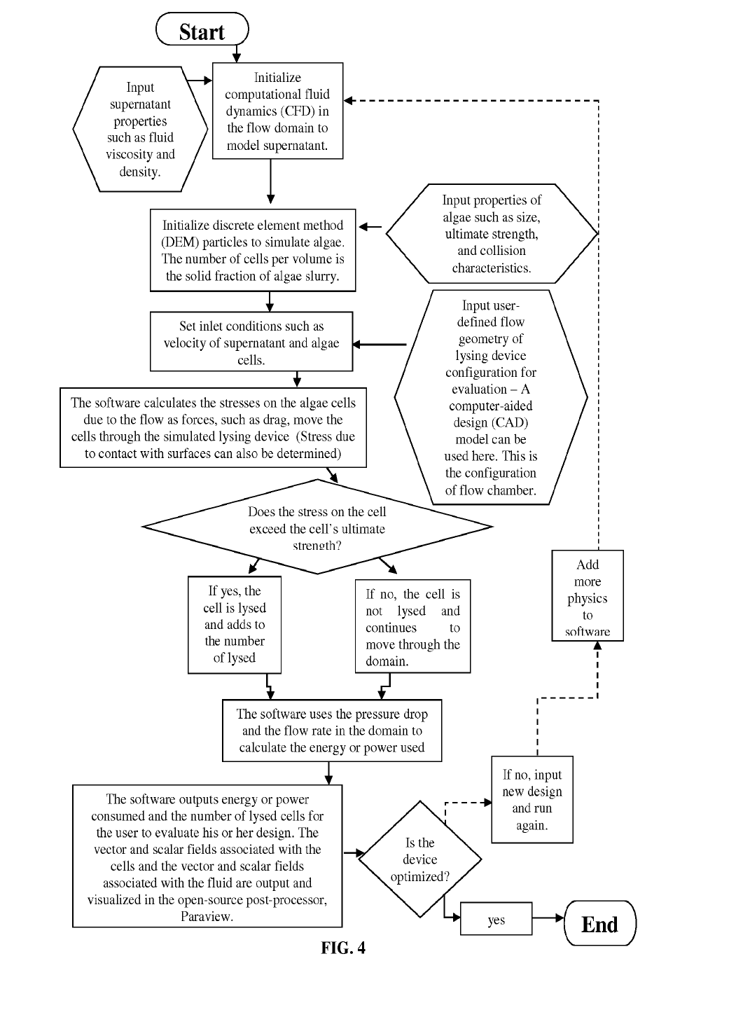

Modeling systems are implemented on a computing device (computer) as processes. In the context of computing, a process is, broadly speaking any computer-implemented activity that generates an outcome, such as implementation of a mathematical or logical formula or operation, algorithm, etc. FIG. 3 illustrates one embodiment of a system 100 for implementing a modeling system. The system 100 may include a device 102 operating under the command of a controller 104. Device 102 may be referred to herein, without limitation, as a computer or computing device. The broken lines are intended to indicate that in some implementations, the controller 104, or portions thereof considered collectively, may instruct one or more elements of the device 102 to operate as described. Accordingly, the functions associated with the modeling methods (e.g., processes, software, programs) described herein may be implemented as software executing in the system 100 and controlling one or more elements thereof. An example of a device 102 in accordance with one embodiment of the present invention is a general-purpose computer capable of responding to and executing instructions in a defined manner. Other examples include a special-purpose computer including, for example, a personal computer (PC), a workstation, a server, a laptop computer, a web-enabled telephone, a web-enabled personal digital assistant (PDA), a microprocessor, an integrated circuit, an application-specific integrated circuit, a microprocessor, a microcontroller, a network server, a Java™ virtual machine, a logic array, a programmable logic array, a micro-computer, a mini-computer, or a large frame computer, or any other component, machine, tool, equipment, or some combination thereof capable of responding to and executing instructions.

In one non-limiting embodiment, system 100 is implemented as a PC. Furthermore, the system 100 may include a central processing engine including a baseline processor, memory, and communications capabilities. The system 100 also may include a communications system bus to enable multiple processors to communicate with each other. In addition, the system 100 may include storage 106 in the form of computer readable medium/media, such as a disk drive, disk, optical drive, a solid state drive, a tape drive, flash memory (e.g., a non-volatile computer storage chip), cartridge drive, and control elements for loading new software. In embodiments of the invention, one or more reference values may be stored in a memory associated with the device 102. Data, such as images produced by the methods and systems described herein may be organized on computer readable media in a database, which is an organized collection of data for one or more purposes, usually in digital form. In one embodiment, any or all software, data, code, processes, controllers, algorithms, instructions, etc. are stored non-transiently on a computer-readable medium.

Embodiments of the controller 104 may include, for example, a program, code, a set of instructions, or some combination thereof, executable by the device 102 for independently or collectively instructing the device 102 to interact and operate as programmed, referred to herein as “programming instructions”. One example of a controller 104 is a software application (for example, operating system, browser application, client application, server application, proxy application, on-line service provider application, and/or private network application) installed on the device 102 for directing execution of instructions. In one embodiment, the controller 104 may be a Windows™ based operating system. The controller 104 may be implemented by utilizing any suitable computer language (e.g., C\C++, UNIX SHELL SCRIPT, PERL, JAVA™, JAVASCRIPT, HTML/DHTML/XML, FLASH, WINDOWS NT, UNIX/LINUX, APACHE, RDBMS including ORACLE, INFORMIX, and MySQL) and/or object-oriented techniques.

In one embodiment, the controller 104 may be embodied permanently or temporarily in any type of machine, component, physical or virtual equipment, storage medium, or propagated signal capable of delivering instructions to the device 102. In particular, the controller 104 (e.g., software application, and/or computer program) may be stored on any suitable computer readable media (e.g., disk, device, or propagated signal), readable by the device 102, such that if the device 102 reads the storage medium, the functions described herein are performed. For example, in one embodiment, the controller 104 may be embodied in various computer-readable media for performing the functions associated with processes embodying the modeling methods.

In one embodiment, the software is written and developed using a programming language, such as the C++ computing language. An integrated development environment (IDE), such as “DevC++, is used to write the code. The developed software generates large text files with data about the flow field (e.g., pressure and velocity), the algae positions in the flow, and the number of lysed algae cells. To visualize the fluid and the algae, a data visualizer is used, such as the opensource Paraview tool from Kitware, Inc. of Clifton Park, N.Y. Finally, our software is setup to take into it an arbitrary computational mesh to represent the occlusion. The occlusion, such as a sphere described below in the examples is created in computer-aided design (CAD) software, such as SOLIDWORKS® from Fisher/Unitech of Troy, Mich. A person of ordinary skill in the art of computer programming and engineering will be able to develop and implement software capable of carrying out the tasks/operations described herein.

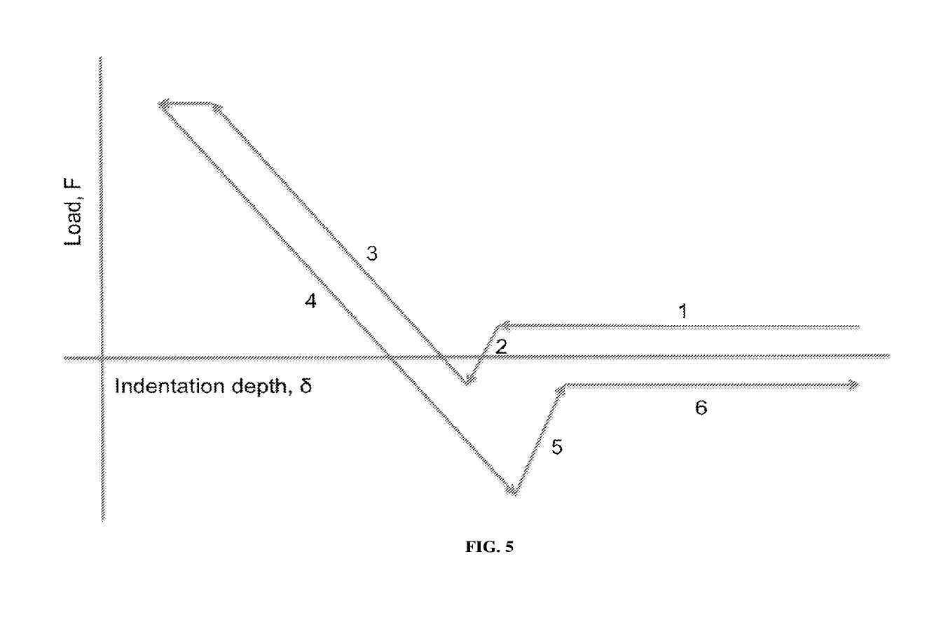

A flow diagram of one embodiment of the computer-implemented method is shown in FIG. 4. The computer-implemented method comprises modeling fluid dynamics within the flow domain of the supernatant. The flow domain is defined geometrically by the walls or lumen of the lysing structure, and includes any flow-altering structure of the lysing structure, such as an occlusion, a restriction, an expansion, a wall roughness or a bending of the flow path. The method also comprises modeling algae cells as discrete elements within the flow domain. The method calculates stresses on the algae cells due to the flow as forces. Data useful for the fluid dynamic modeling include fluid viscosity, density and flow rate. Fluid viscosity and density is approximated in one embodiment by density and viscosity values for fresh water (e.g., 992-1000 kg/m³ and 0.0007-0.0013 Pa-s) or salt water (e.g., 1028-1013 kg/m³ and 0.0006-0.0015 Pa-s), depending upon the growth medium of the algae cells. Algae properties include, without limitation, diameter (e.g., length, width, and height), density, volume, velocity and drag. Properties of the lumen or internal surface of the lysing structure through which the fluid flows, in addition to geometric features includes friction or drag, which can significantly impact fluid dynamics. The “strength,” that is, the stresses needed to rupture or lyse the algae cells also is relevant when modeling the degree of lysis of algae in an algae culture. By including the strength of the cells, flow regions where stresses exceed that threshold can be mapped and maximized. Table A, above, provides examples of bursting strength of certain cell types.

According to another embodiment, a non-transitory computer readable medium having stored thereon instructions is provided. The instructions, when executed by a processor, cause the processor to implement a computer-implemented method as described above, for optimizing lysis of cells in a lysing structure.

### EXAMPLES

### Example 1

### Atomic Force Microscopy to Probe Microalgal Elastic Response

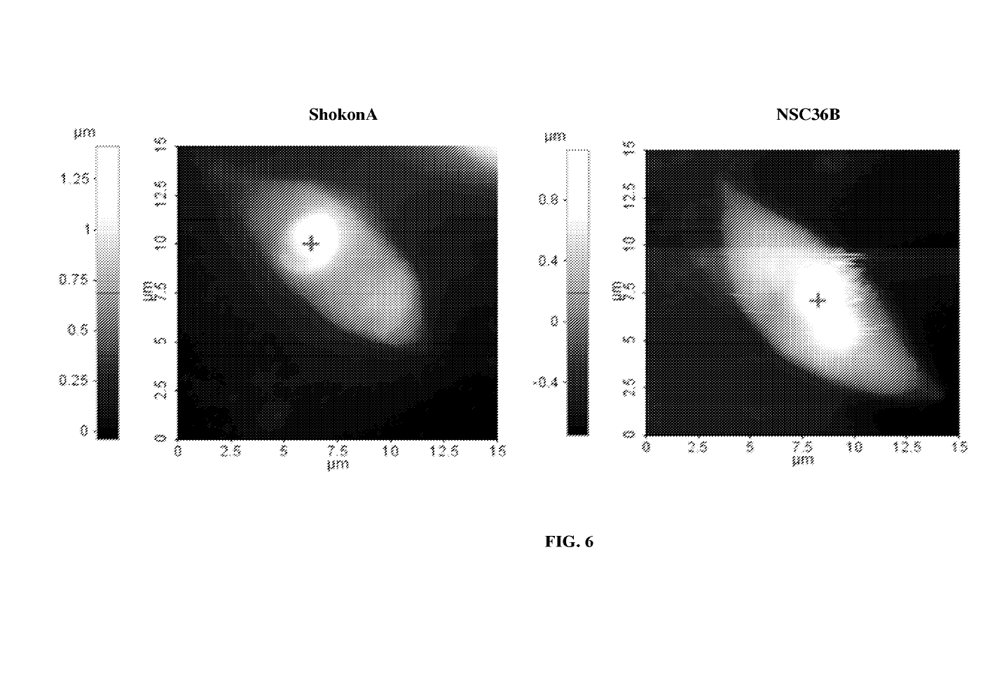

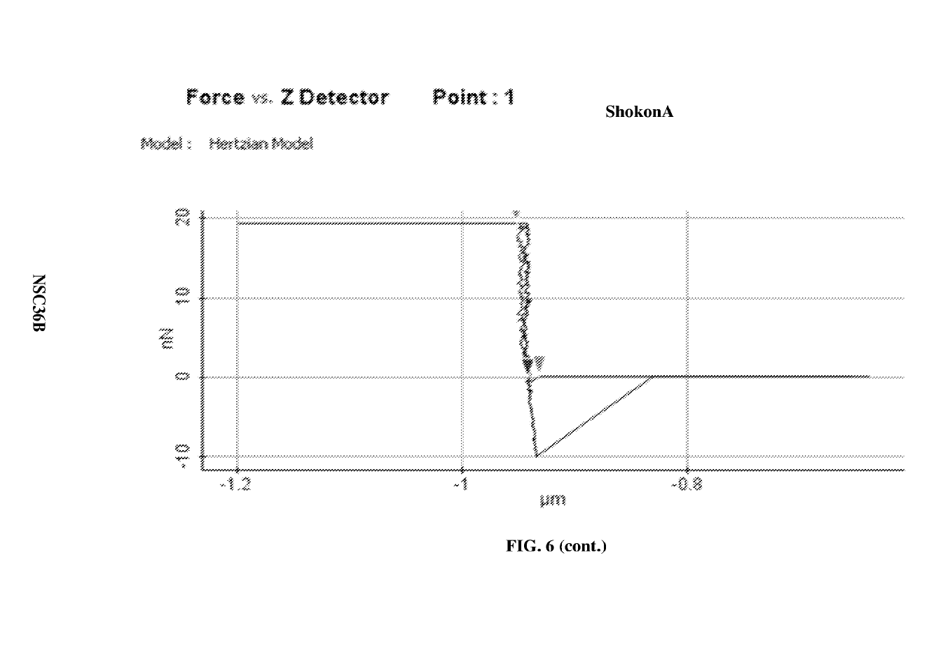

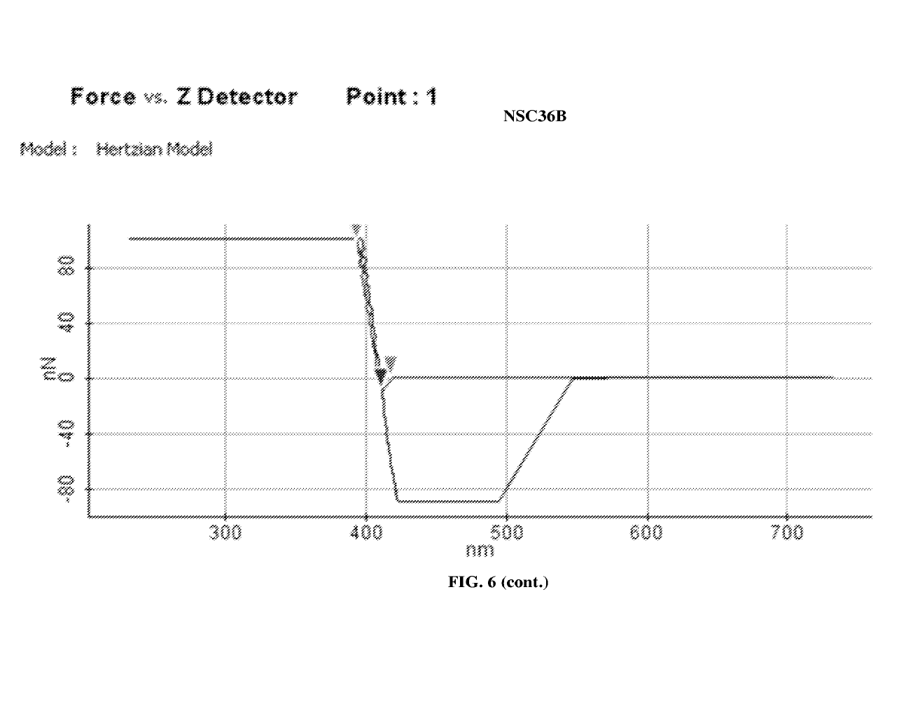

The mechanical properties of microalga, Scenedesmus dimorphus, were investigated through Atomic Force Microscope (AFM) to determine the elastic response of cells that were in an aqueous and dried state using local nano-scale indentation. We determined the Young's Modulus of single-celled S. dimorphus cells to be 2.21±0.40 MPa when in an aqueous state and 57.96±7.20 MPa in a dried state. FIG. 5 shows a force/distance curve generated by an AFM. FIG. 6 shows images of S. dimorphus on an AFM and respective force/distance curves.

We used the microalgal strain Scenedesmus dimorphus, which has many applications including its use in biomass and wastewater treatment applications. (P. Chevalier and J. de la Noue, “Wastewater nutrient removal with microalgae immobilized in carrageenan,” Enzyme and Microb. Technol., vol. 7, no. 12, pp. 621-625, 1985) While present in freshwater, S. dimorphus can also grow in domestic sewage. These variations of growing conditions can cause phenotypical changes allowing these 10 μm long microorganisms to grow in colonies or alone in crescent, bi-convex, rounded tetrad, or circular shapes. (G. Oron, G. Shelef and A. Levi, “Environmental phenotypic variation of Scenedesmus dimorphus in high-rate algae ponds and its relationship to wastewater treatment and biomass production,” Biotechnology and bioengineering, vol. 23, no. 10, pp. 2185-2198, 1981) The molecular composition of S. dimorphus varies as well such as for protein, carbohydrates, and lipids are 8-18%, 21-52%, and 16-40% by dry weight, respectively. (T. Bruton, H. Lyons, Y. Lerat, M. Stanley and M. B. Rasmussen, “A review of the potential of marine algae as a source of biofuel in Ireland,” Sustainable Energy Ireland, Dublin, 2009).

To probe the mechanical properties of these microorganisms, atomic force microscope (AFM) was employed, which enabled high resolution imaging as well as force measurements when obtaining mechanical properties. (Y. F. Dufrene, “Atomic force microscopy, a powerful tool in microbiology,” Journal of Bacteriology, vol. 184, no. 19, pp. 5205-5213, 2002) Mechanical properties, like elastic modulus, of various biological materials have been obtained through AFM previously including that for mouse embryonic stem cells and yeast cells, which have elastic moduli values of 17.87 kPa and 0.6 MPa, respectively. (A. J. Heim, W. G. Matthews and T. J. Koob, “Determination of the elastic modulus of native collagen fibrils via radial indentation,” Applied Physics Letters, vol. 89, no. 18, 2006; H. Ladjal, J.-L. Hanus, A. Pillarisetti, C. Keefer, A. Ferreira and J. P. Desai, “Atomic force microscopy-based single-cell indentation: experimentation and finite element simulation,” in IEEE, St. Louis, 2009; and A. Touhami, B. Nysten and Y. F. Dufrene, “Nanoscale mapping of the elasticity of microbial cells by atomic force microscopy,” Langmuir, vol. 19, no. 11, pp. 4539-4543, 2003).

We cultured S. dimorphus (UTEX algae culture collection) in Modified Bold 3N and Proteose media. For AFM imaging, glass slides were plasma cleaned and for the aqueous experiments, they were treated with 0.01% poly-1-lysine solution to enhance cell adhesion. The dried samples were inoculated from the culture grown on proteose agar medium and allowed to dry on the slides. Both imaging and nanoindentation on the S. dimorphus cells were performed on an Asylum Research MFP-3D AFM under ambient air at room temperature. Two types of cantilevers were used in the probing of the hydrated and dehydrated S. dimorphus cells, having spring constants ranging from 0.08-0.26 N/m and 0.5-4.4 N/m, respectively. The specific spring constants for the cantilevers used were calculated using the Sader method (J. E. Sader, J. W. M. Chon and P. Mulvaney, “Calibration of rectangular atomic force microscope cantilevers,” Review of Scientific Instruments, vol. 70, no. 10, pp. 3967-3969, 1999) implemented with the Asylum Research software.

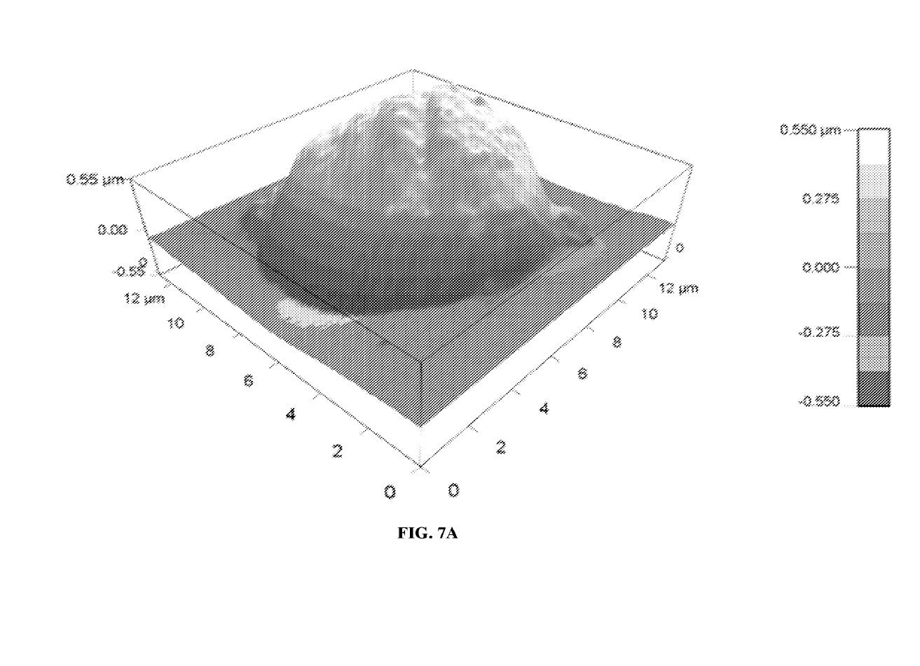

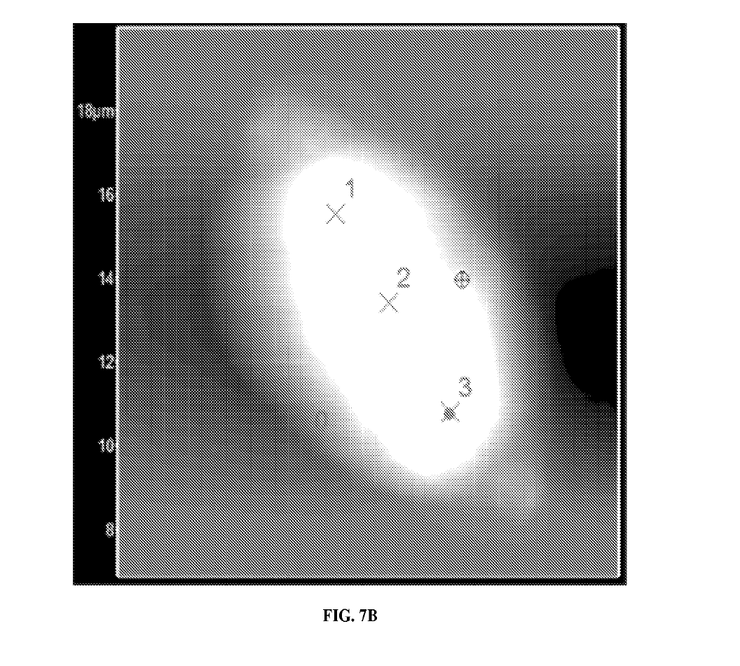

FIG. 7A shows a representative AFM 3D height image showing the surface topography of a dried S. dimorphus cell. FIG. 7B shows AFM topographical image of a typical single, dried S. dimorphus cell with points 1, 2, and 3 selected for nanoindentation to determine the Young's Modulus.

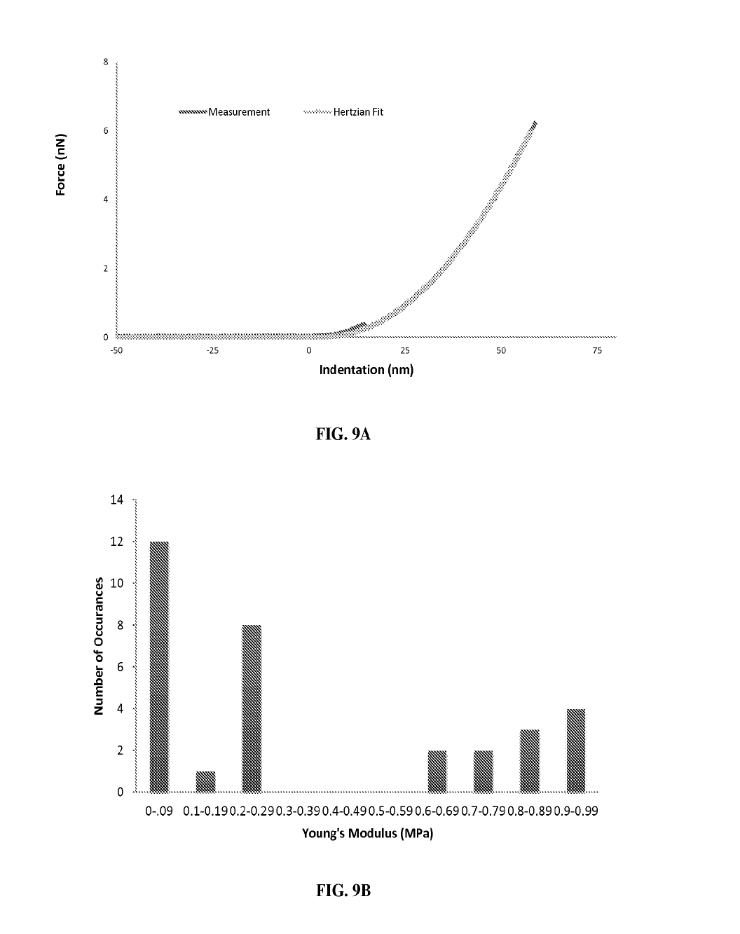

Before nanoindentation, a single, dried cell was imaged in air using the stiffer cantilever to obtain the representative 3D height image shown in FIG. 9A. Next, the nanoindentation was initiated at select points on the cell to determine the mechanical properties (FIG. 9B). A minimum of ten measurements was taken at each location. Utilizing Igor Pro software, approach data from these indentations were analyzed using relationships for a Hertzian contact model, which has previously implemented. (H. Ladjal, J.-L. Hanus, A. Pillariseti, C. Keefer, A. Ferreira and J. P. Desai, “Atomic force microscopy-based single-cell indentation: experimentation and finite element simulation,” in RSJ International Converence on Intelligent Robot Systems, St. Louis, 2009).

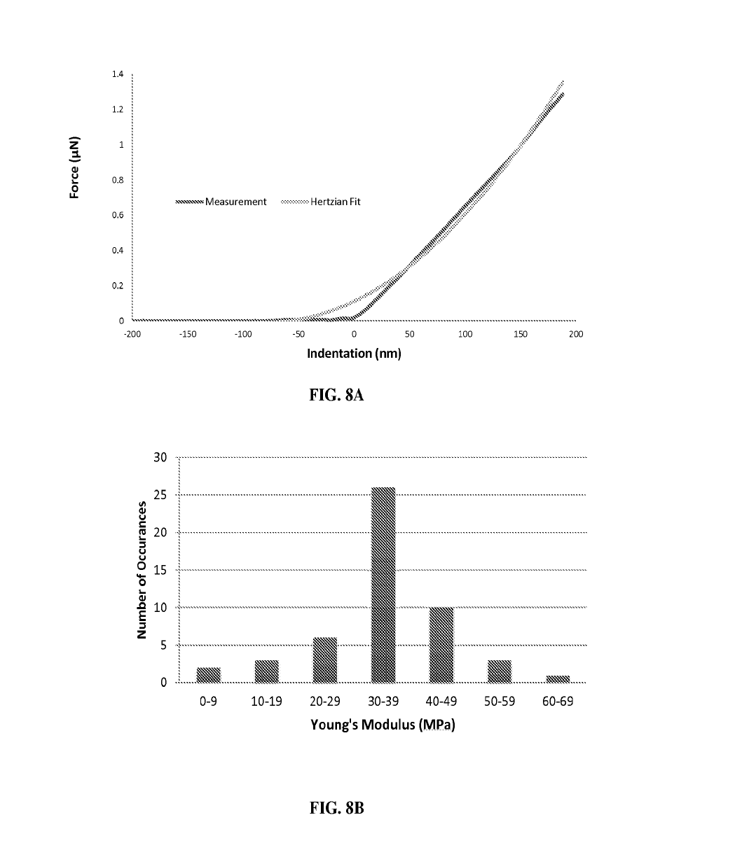

A representative force vs. indentation plot with the Hertzian contact model approximation applied is shown in FIG. 8A. After accumulating the force-indentation data from the three points of a cell, histogram plots were determined to project the variation in the data (FIG. 8B). This histogram shows a normal distribution of Young's modulus values with a mean value of 36.72 MPa with a standard distribution of 10.71 MPa.

FIG. 8A shows a representative AFM force vs indentation approach curve from a defined nanoindentation location on single, dried S. dimorphus cell. In this experiment, the Young's modulus was calculated to be 26.85 MPa using a Hertzian contact model. FIG. 8B shows a histogram of binned Young's Modulus values from the designated locations on the same cell.

For cells in solution, a representative force versus indentation curve is shown in FIG. 9A. The results have a close fit when implementing the Hertzian approximation resulting in a Young's modulus of approximately 3.95 MPa. Similar to the dried cells, a histogram plot was generated from the multiple nanoindentations on single dried cell (FIG. 9B). The histogram of dried cell Young's modulus values appear to be more broadly distributed, which may be due to the differences in viscoelastic properties that may be more prevalent when they are in solution versus dried.

FIG. 9A shows a representative AFM force vs indentation approach curve for a S. dimorphus cell in solution. The Young's modulus here was calculated to be 3.95 MPa using a Hertzian contact model assumption. FIG. 9B shows a histogram of binned Young's Modulus values from the representative locations on the same cell.

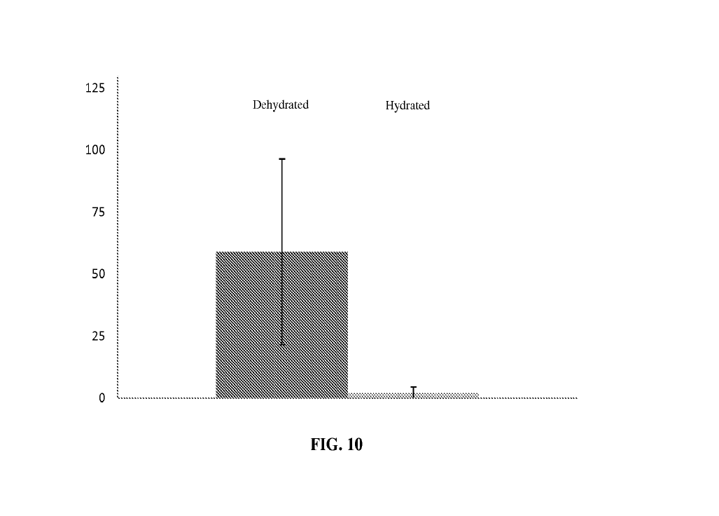

After analyzing the results from both cell types, the Young's moduli values were calculated with 95% confidence intervals (p≦0.05) for S. dimorphus. For the dried cells, the Young's modulus was calculated to be 57.96±7.20 MPa (mean±standard deviation of the mean) with a standard deviation (or data spread) of 36.55 MPa. The Young's modulus value for aqueous S. dimorphus cells is 2.21±0.40 MPa with a standard deviation of 2.25 MPa (FIG. 10).

FIG. 10 shows a comparison of dried and aqueous Young's modulus measurements of Scendesmus dimorphus cells. The error bars represent standard deviation with p≦0.05.

In conclusion, we were able to both image and determine the elastic properties for Scenedesmus dimorphus cells using an AFM as both dried and in solution with a result of 57.96±7.20 MPa and 2.21±0.40 MPa, respectively. In addition, comparing the dry and aqueous states for algae shows a significant difference in their elastic properties. These findings may be particularly important for cell lysis especially for the diversity of industries where this may be applicable including dietary supplements, cosmetics, pharmaceuticals, and biofuel. We believe that these findings will be important to researchers in physics, biology, engineering, and chemistry.

### Example 2

One step in processing algae for biofuel is successfully rupturing the algae cell so that it can release its lipid content, the main energy component of the cell. Currently, this process is very energy-intensive, thus commercial, algal biofuel usage is on halt. To obtain a better understanding this process, mechanical characterization of the microalgal cell must be performed so that mechanical lysing can be analyzed.

With this work, we hope to bring a greater understanding of the mechanical properties of microalgae in order to make algal biofuel processing more efficient. Using an Asylum Research MFP-3D AFM, we have imaged algal strain S. dimorphus and determined its Young's modulus in both its hydrated and dehydrated forms, 2.21±0.40*MPa and 57.96±7.20*MPa respectively. The Young's modulus is necessary for computational modeling evaluated with a Particle Flow and Tribology Laboratory in-house code. This model would allow us to further optimize microalgal cell lysis for biofuel processing.

In addition to our experimentation with atomic force microscopy, we have also proven that shear stress aids in the lysis of microalgal cells utilizing a Tecan Safire II spectrophotometer. Using Bodipy 505/515, a green lipophilic fluorescent dye, we were able to stain the lipid components of the cells and perform various cell disruption techniques including microwave, sonication, and shearing between glass and aluminum surfaces. Once the disruption was concluded, we filtered the solutions and used the spectrophotometer to determine the absorbance Bodipy 505/515 remaining in the precipitate. This revealed that shearing worked significantly better than the other methods employed.

### Calculating Young's Modulus

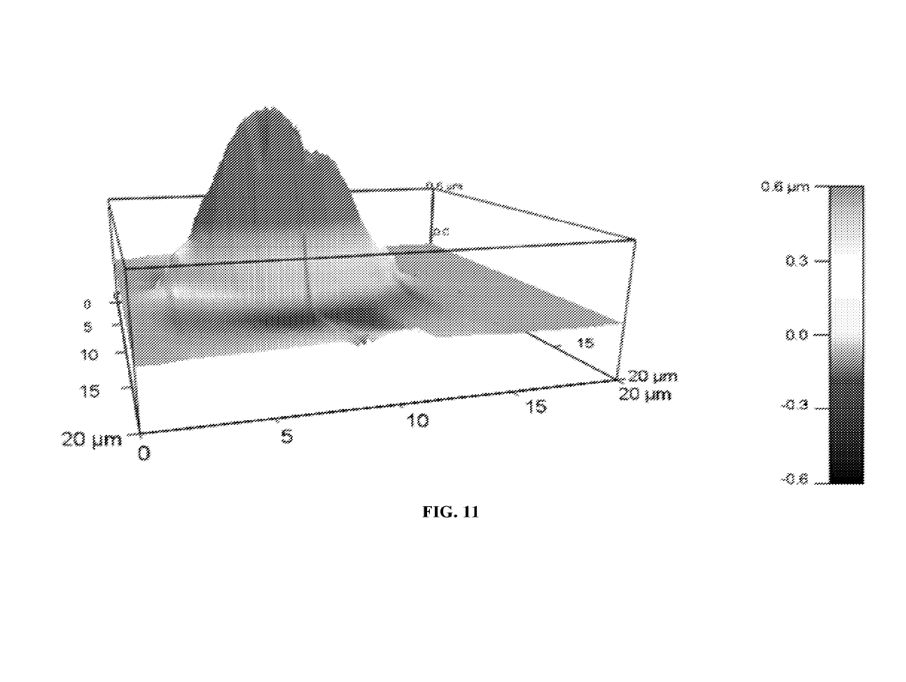

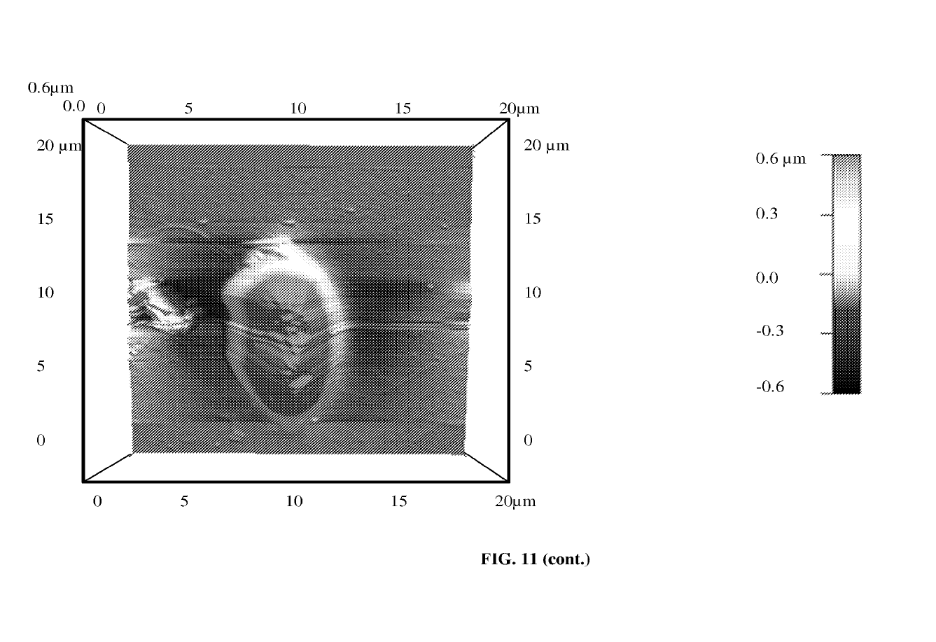

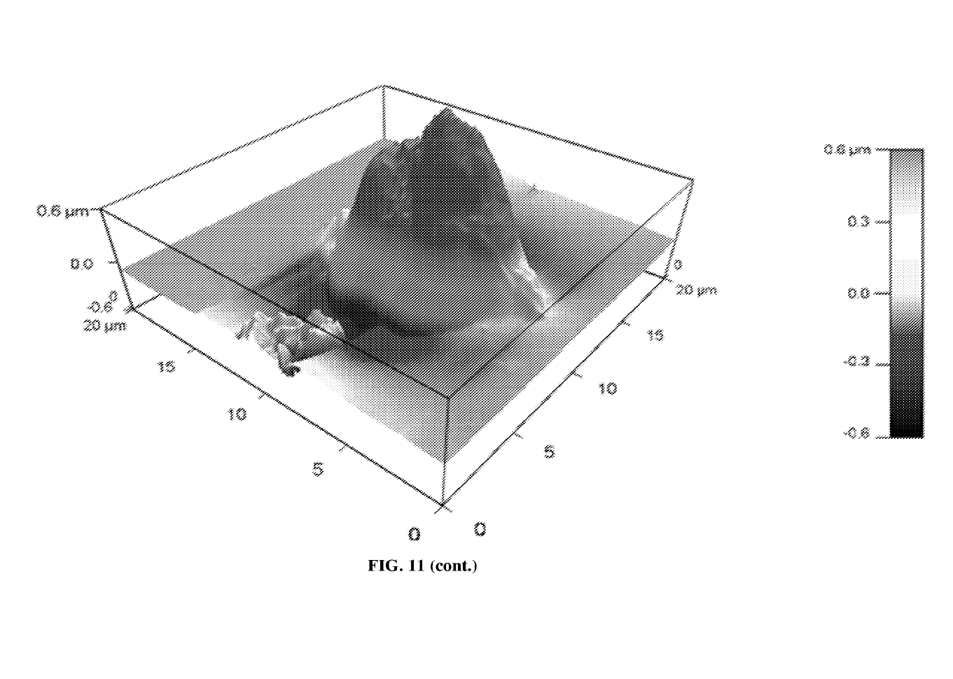

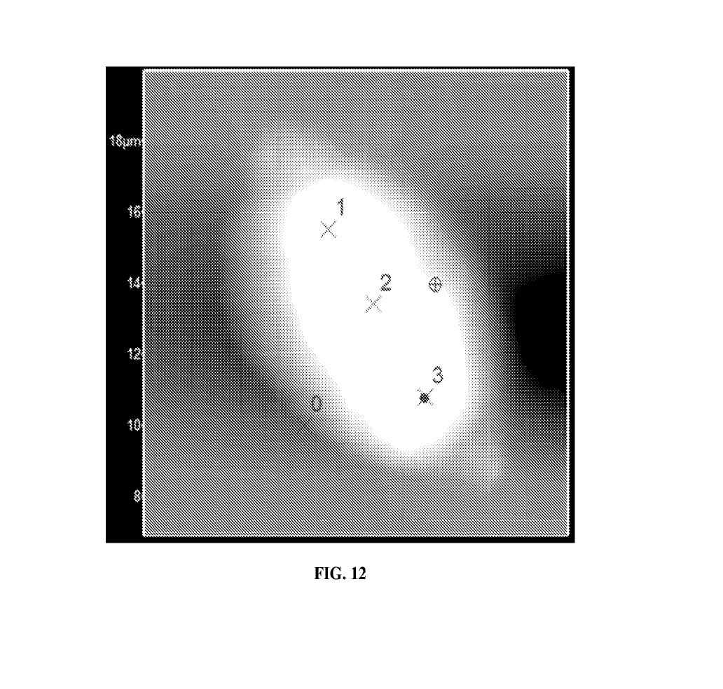

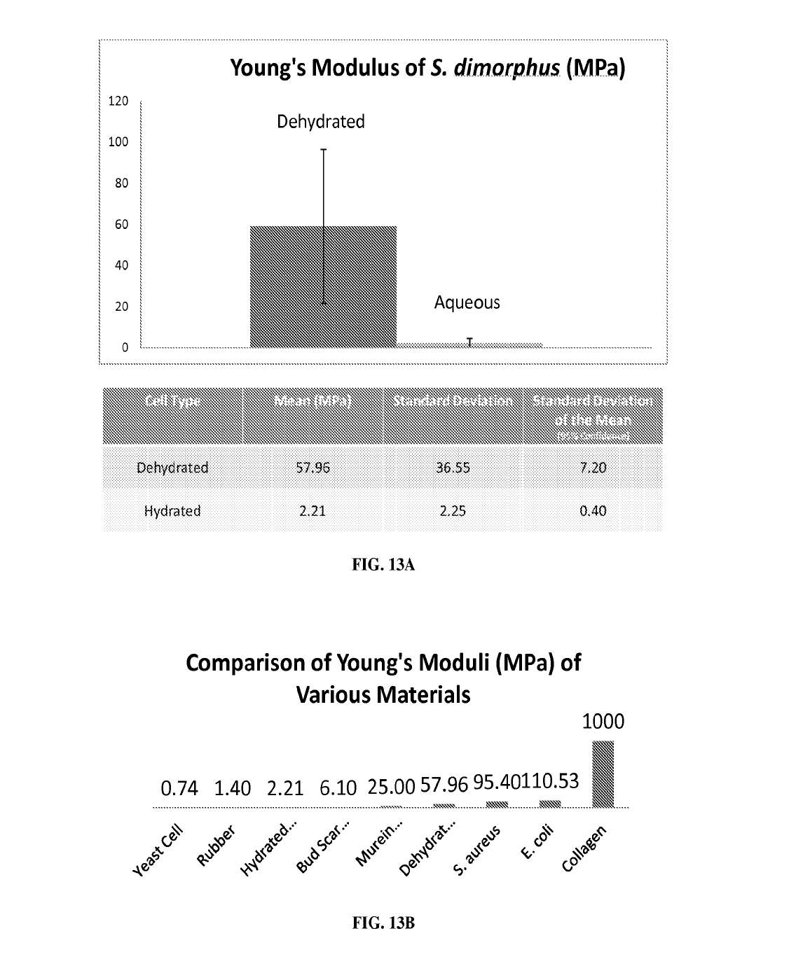

3D images of a single dried S. dimorphus cell were taken and are shown in FIG. 11. FIG. 12 shows topography image from Asylum Research MFP-3D-AFM with points selected for indentation. The cell shown in FIG. 12 is the same cell as shown in FIG. 11. Young's Modulus was obtained, as shown in FIG. 13A. FIG. 13B provides young's moduli for various materials ([1] Radmacher, M., “Measuring the Elastic Properties of Biological Samples with the AFM,” IEEE Engineering in Medicine and Biology March/April 1997, pp. 47-57; [2] Eaton, P., et al. “Atomic force microscopy study of the antibacterial effects of chitosans on Escherichia coli and Staphylococcus aureus,” Ultramicroscopy 108 (2008) 1128-1134; [3] Tuson, H. H., et al. “Measuring the stiffness of bacterial cells from growth rates in hydrogels of tunable elasticity,” Molecular Microbiology (2012) 84(5), 874-891; [4] Felling, A. E., et al. Local Nanomechanical Motion of the Cell Wall of Saccharomyces cerevisiae,” Science 305, 1147 (2004); [5] Touhami, A, et al., Nanoscale Mapping of the Elasticity of Microbial Cells by Atomic Force Microscopy,” Langmuir 2003, 19, 4539-4543).

### Disruption Tests

### Preparation

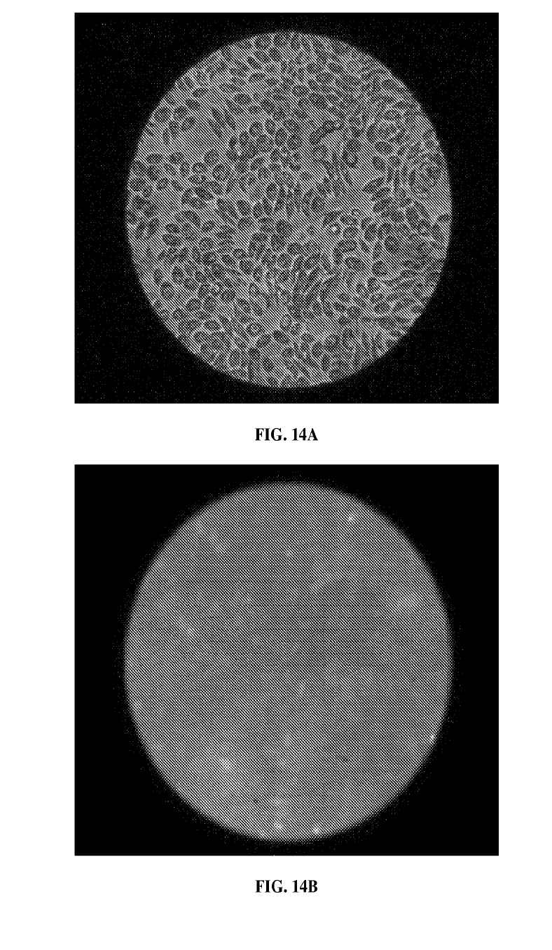

Algae were allowed to absorb dye for approximately 1 week. Then the viability of the dye was checked by examining the fluorescence. The algae and dye were spun down and the dye solution was replaced with dH₂O. The same exposure (0.3) was kept throughout for imaging for consistency. The magnification was increased to 63× (2.5× was on for a total 157.5×). FIG. 14 shows S. Dimorphus with Bodipy dye under bright field (FIG. 14A) and fluorescence (FIG. 14B) microscopy. This microscopy was undertaken to ensure that the cells absorbed the Bodipy dye.

### Disruption Methods

There were four disruption methods investigated to compare their effectiveness: sonication, frothing, microwave, and manual shearing via metal flat punch. The microalgae slurry was placed in a sonicator for 14 minutes (as described in the literature). The algae solution is poured into 80 mL beaker for the frothing disruption test. A frother was used on the microalgae slurry for 7 minutes. The microwave was utilized for a total of 7 minutes as well. However, due to the evaporation of the dH₂O, this disruption method required 3 times more microalgal slurry to obtain enough of a sample to be used in the analysis via spectrophotometry. The fourth disruption method used was shear via a metal flat punch whose diameter was approximately 1.5 inches. The slurry was sheared between the flat punch and a glass petri dish for 7 minutes.

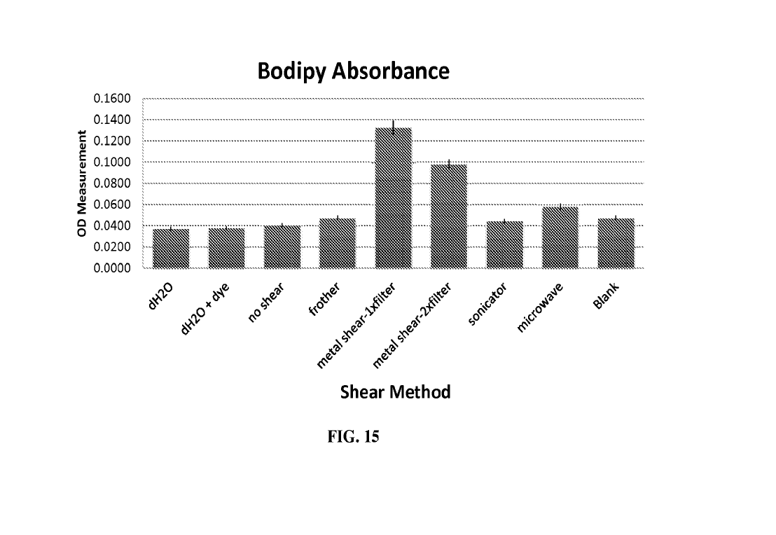

The microalgae solutions were removed from their respective disruption tests with needle and syringe and a drop of each sample was placed on a coverslip. A 0.22 μm filter was used for the remainder of the tested microalgal slurry to remove intact cells and debris before measurement. The microalgal slurry obtained from the flat punch shear test was filtered twice to ensure only subcellular parts were used in the following optical density tests. Optical density measurements were undertaken with a Tecan Safire Photospectrometer (Tecan, Mannedorf, SU). The Bodipy dye used to stain the algal lipids becomes excited between 450-490 nm. Three controls were included: 1.5 ml dH₂O to 0.5 μl Bodipy; dH₂O; and blank wells within 96-well microplate. The results (FIG. 15) show that shearing is a good candidate for algal cell lysing.

### Example 3

### Prediction of Stress Values Suitable to Lyse Cells in Cell Lysis Experiments

In this document, a quick study is presented on the methods used in the software to achieve stresses which are equivalent to stress values from the literature which are known to lyse cells. The details of the software have been provided in previous documents. Here, only the methods used to achieve these stresses and the resulting velocity field and pressure field in the flow will be discussed. Cell lysis by fluid shear is can be accomplished by subjecting cells to a flow with sufficient shear stress to rupture their membrane (Born et al., 1992). This was accomplished by Born et al. in 1992 who used a viscometer to impart known shear stresses to animal cells. Born et al. subjected the animal cells to flows with shear stress values between 124 N/m² and 577 N/m² and recorded the percentage of cells which were lysed. They also compared this value to an analytical model they developed and found very good agreement.

This data from Born et al. indicates that 200 N/m² is sufficient to lyse approximately 45% of the cells they tested. As such, in the current software it was desired to achieve shear stress values of 200 N/m² to provide evidence that the software, and the device configuration, are capable of producing stress values large enough to lyse actual cells. To accomplish this goal, the software was configured in the same manner as in previous studies. To produce large stress values, the inlet flow velocity was set to 2500 mm/s. As the fluid moved through the domain, it accelerated and sheared due to the occlusion. The resulting shear stress contour plot and quantitative stress values are displayed in FIG. 16.

In FIG. 16, it is clear that the software can predict stress levels in excess of 200 N/m². As a result of the experimental data provided by Born et al., it is believed that cells placed in the simulated flow in regions of stress in excess of 200 N/m² would be lysed.

To get a better understanding of the flow field, the pressure and the velocity in the domain are provided in FIG. 17, which shows flow characteristics in the device domain. FIG. 17A shows a contour plot of the velocity field. FIG. 17B shows a contour plot of the pressure field. In FIG. 17, velocity contour and pressure contour plots are displayed. From FIG. 17A, it is clear to see that the flow accelerates as it moves past the occlusion. In FIG. 17B, the pressure drop in the domain is displayed. In this work, evidence is provided that to show that software can predict stresses suitable for lysing cells.

### Example 4

### A Method of Predicting Energy Consumption During Cell Disruption and Lipid Extraction for Algae-Based Biofuel Production

In the current work, a computational framework is proposed to predict the energy consumed during mechanical cell disruption for algae-based biofuel production. Fluid-induced shear stress applied to the cell is simulated by computational fluid dynamics (CFD). The motion of the individual algae cells is simulated with the discrete element method (DEM). The energy consumed and lysing efficiency are recorded. The model predicted that as energy consumption is increased, more of the algae cells in the domain are disrupted. It was also found that the model can be used to predict the areas in the flow where lysing is most likely to occur. Such information can be used to design novel cell disruptor configurations which minimize energy consumption.

### Methodology

To predict fluid-induced algae cell lysis, it is important to model both the fluid and the algae cells. For the fluid, an in-house numerical solver is developed. To simulate the algae cells, a Lagrangian particle modeling approach is employed. In this section, the details for each technique are provided. Data was generated using custom-developed in DevC++. Data was visualized in Paraview. Geometric structures were created in SOLIDWORKS®.

### Supernatant Modeling

The supernatant, in which the algae grow, consists primarily of water and a small amount of nutrients. In practice, it is believed that this fluid can be used as the medium to induce lysis for lipid release by imparting shear stresses on the algae cell. In the current framework, the supernatant is modeled using computational fluid dynamics (CFD). The Chorin projection is used to numerically approximate the Navier-Stokes equations (Chorin, A. J., Numerical solution of the Navier-Stokes equations. Mathematics of Computation 1968, 22, (104), 745-762 and Griebel, M. et al., Numerical Simulation in Fluid Dynamics. Society for Industrial and Applied Mathematics (SIAM): Philadelphia, 1998; p 217). Beginning with the momentum equations, (1a), (1b), and (1c), an explicit Euler time-stepping algorithm is used to solve for the new velocity components, on a staggered grid, at each successive time-step. The variables u,v,w, and p represent the x,y,z components of the fluid velocity and the pressure, respectively.

$$\begin{matrix}ρ ( \frac{\partial u}{\partial t} + u \frac{\partial u}{\partial x} + v \frac{\partial u}{\partial y} + w \frac{\partial u}{\partial z} ) = - \frac{\partial p}{\partial x} + μ ( \frac{{\partial }^{2} u}{\partial {x}^{2}} + \frac{{\partial }^{2} u}{\partial {y}^{2}} + \frac{{\partial }^{2} u}{\partial {z}^{2}} ) & ( 1 a ) \\ ρ ( \frac{\partial v}{\partial t} + u \frac{\partial v}{\partial x} + v \frac{\partial v}{\partial y} + w \frac{\partial v}{\partial z} ) = - \frac{\partial p}{\partial y} + μ ( \frac{{\partial }^{2} v}{\partial {x}^{2}} + \frac{{\partial }^{2} v}{\partial {y}^{2}} + \frac{{\partial }^{2} v}{\partial {z}^{2}} ) & ( 1 b ) \\ ρ ( \frac{\partial w}{\partial t} + u \frac{\partial w}{\partial x} + v \frac{\partial w}{\partial y} + w \frac{\partial w}{\partial z} ) = - \frac{\partial p}{\partial z} + μ ( \frac{{\partial }^{2} w}{\partial {x}^{2}} + \frac{{\partial }^{2} w}{\partial {y}^{2}} + \frac{{\partial }^{2} w}{\partial {z}^{2}} ) & ( 1 c ) \\ \frac{\partial u}{\partial x} + \frac{\partial v}{\partial y} + \frac{\partial w}{\partial z} = 0 & ( 2 )\end{matrix}$$

Pressure-velocity coupling is achieved by satisfying the continuity equation (Eq. (2)). The pressure at each time-step is solved through the successive over-relaxation (SOR) method.

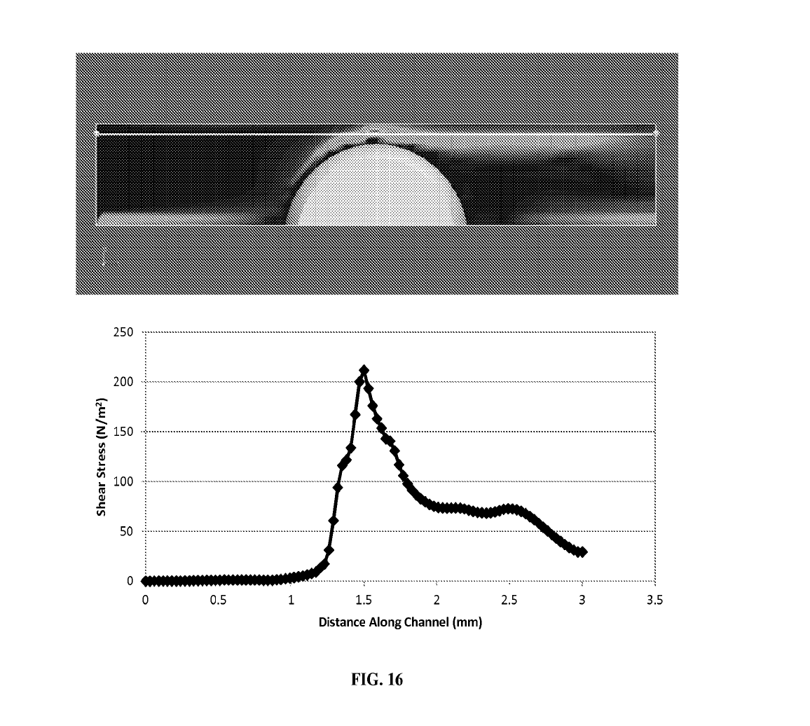

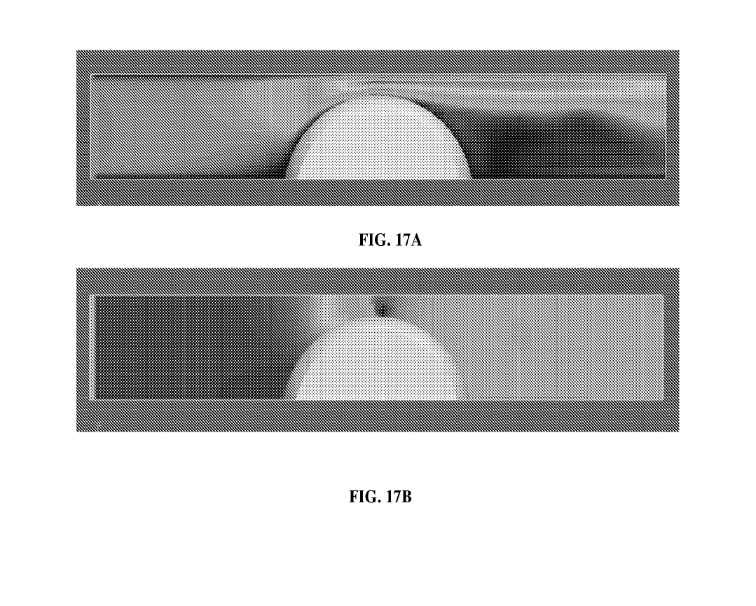

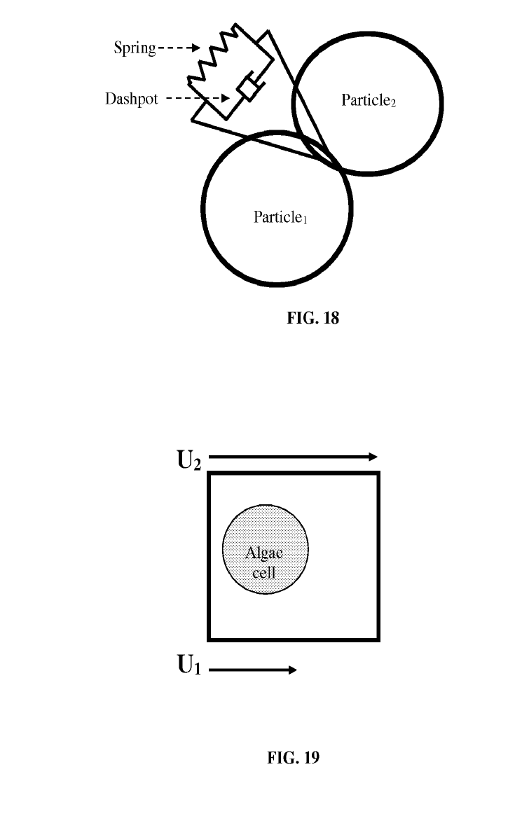

Algae Modeling The algae are modeled using the discrete element method (DEM). DEM was first developed by Cundall and Strack in 1979 and has been used for a number of particle modeling applications (Mpagazehe, J. N.; Queiruga, A. F.; Higgs, C. F., Towards an understanding of the drilling process for fossil fuel energy: A continuum discrete approach. Tribology International 2012, Article in Press; Zhang, H.; Tan, Y.; Yang, D.; Trias, F. X.; Jiang, S.; Sheng, Y.; Oliva, A., Numerical investigation of the location of maximum erosive wear damage in elbow: Effect of slurry velocity, bend orientation and angle of elbow. Powder Technology 2012, 217, 467-476; Cundall, P. A.; Strack, O. D. L., Discrete Numerical Model for Granular Assemblies. Geotechnique 1979, 29, (1), 47-65; and Rojek, J., Discrete Element Modelling of Rock Cutting. Computer Methods in Materials Science 2007, 7, (2), 224-230)⁷⁻¹⁰. In DEM, particles are represented as discrete entities with properties such as radius, mass, position, and velocity. When it is found that the particle centers are at a distance less than the sum of their radii, a collision is performed. To calculate the forces on the particles after a collision, the classic spring-dashpot model is used. This model is illustrated in FIG. 18. In FIG. 18, a collision between two particles is depicted. When Particles collides with Particel₂, Eq. (3) is used to resolve the forces on the particles from the collisions.

F_{collison}=K_{spring}U_{spring}U_{spring}−Vₙ−B_{dashpot} (3)

In Eq. (3) K_{spring} and B_{dashpot}, the spring and damping constants respectively, are specified depending on whether the collision is with other particles or with the boundaries. The numerical integration scheme used to update particle velocities and positions is the explicit Euler integration technique. In the current model, the spring constant and damping parameters are set to zero. Experimental investigations can be performed to provide an appropriate estimate for these parameters for algae.

### Fluid Forces on Algae

As the supernatant flows around the algae, it exerts forces on it. In the current model, the forces from the supernatant serve two purposes; (1) the forces from the supernatant act to advect algae cells through the domain, and (2) the forces from the supernatant exert shear stresses on the algae which, if larger than the algae shear strength, will cause the algae cell membrane to rupture. A DEM particle, representing an algae cell, surrounded by a CFD element is depicted in FIG. 19. In FIG. 19, the velocity of the supernatant below the algae cell is denoted as U₁. The velocity of the supernatant above the algae cell is denoted as U₂. The drag forces which advect the algae cell in the domain are calculated from Stokes drag (Eq. (4)).

F_{drag}=3πηdU (4)

In Eq. (4), η is the viscosity of the supernatant, d is the diameter of the algae cell, and U is the relative velocity between the fluid and the cell. In the instance depicted in FIG. 19, U₁ for Eq. (4) is calculated as the difference between the algae cell's velocity and the average of U₁ and U₂. As the flow moves around the algae cell, it imparts shear stresses on the algae cell's surface. A portion of this shear stress acts to move the algae cell in the domain (drag). For simplicity, the shear stress which contributes to cell disruption is calculated independently from the drag and is based upon the velocity gradient across the algae cell. This calculation is displayed in Eq. (5).

$$τ = \frac{6 η ( {U}_{2} - {U}_{1} )}{( d )} \qquad ( 5 )$$

In Eq. (5), τ represents the shear stress on the cell. The formulation is derived by assuming a Stokes drag force acting on half of a cell due to the velocity gradient across the cell. Though this formulation captures the velocity gradient across the cell, which is the dominant phenomenon contributing to the shear stress on the cell, a higher fidelity approximation can be incorporated into the model at a later date. However, for the purpose of modeling physics-based algae lysing with the current framework, Eq. (5) suffices as a means to simulate this behavior. In the model, a shear strength for the algae is prescribed. As the simulation runs, τ is calculated at each time-step. If τ is found to be larger than the prescribed algae shear strength, then the algae are determined to have been lysed.

### Calculating Power Consumption

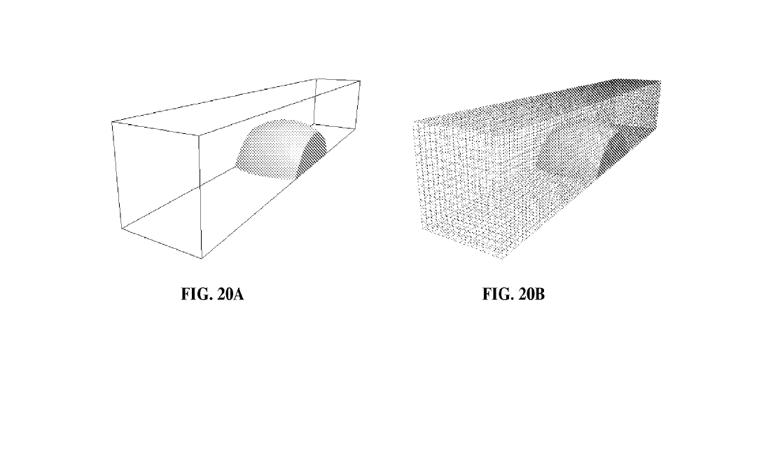

Because the velocity gradient across the algae cell is important in calculating the shear stress being applied to it, an obstacle was placed in the flow to generate different velocity gradients. The computational domain with the obstacle is displayed in FIG. 20A. In FIG. 20B, the computational mesh for the CFD is displayed. In FIG. 20A the fluid domain is shown as an outline around the obstacle placed in flow, and in FIG. 20B the fluid domain is shown as a wireframe to display CFD mesh.

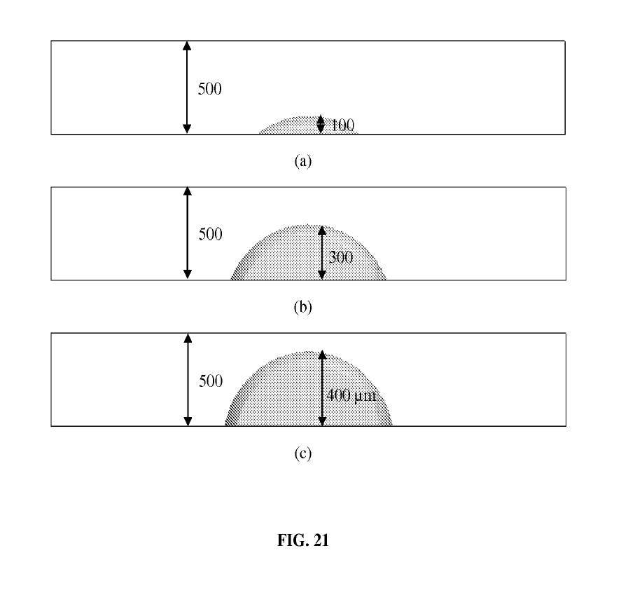

In this work, the occlusion ration, φ, is defined as the ratio of the height of the domain that is blocked to the total height of the domain. This parameter is varied by moving the obstacle in the flow to higher or lower positions which occupy different portions of the domain. When φ is large, it means a large portion of the flow is occluded by the obstacle. Conversely, when φ is small, it means a small portion of the flow is occluded by the obstacle. In FIG. 21, the effect of varying this parameter is depicted. The occlusion ratios are set to 0.2 (FIG. 21A), 0.6 (FIG. 21B), or 0.8 (FIG. 21C). The power consumption is calculated as the power it would take for a pump to maintain the specified flow rate in the domain. This is calculated as the product of the pressure drop across the length obstacle, ΔP, and the volumetric flow rate, Q. This relationship is displayed in Eq. (6).

Power Input=ΔP*Q (6)

Changing the occlusion ratio affects the pressure drop and therefore the power input necessary to maintain the constant flow rate, Q. However, changing the occlusion ratio also affects the local velocity gradients across the computational cells, or strain-rates, which affects the stress imparted to the algae. In this way, the power consumption and the algae cell disruption are both predicted in the current framework.

### Results and Discussion

In this section, the results from the model are presented. A series of simulations were run in which the occlusion ratio was varied between 0 and 0.8. The algae were seeded in random locations, and each occlusion ratio was run 3 times. The power consumed for each occlusion ratio and the percentage of algae lysed is reported. In Table 1, the input parameters used in the model are presented.

TABLE 1
Model Input Parameters

| Algae Properties | |
|---|---|
| Diameter (μm) | 30.0 |
| Density (kg/m³) | 1000.0 |
| Supernatant Properties | |
| Viscosity (Pa-s) | 0.001 |
| Density (kg/m³) | 1000.0 |
| Algae Shear Cell Parameters | |
| Length (μm) | 3000.0 |
| Width (μm) | 500.0 |
| Height (μm) | 500.0 |
| Inlet Velocity (μm/s) | 25000.0 |

Only moderate consideration was used to reconcile the properties of the algae and the supernatant to properties observed in experiments. However, the algae size and the supernatant viscosity are two critical parameters in the simulation and their values of 30 microns and 0.001 Pa-s, respectively, are close to actual values found in experiments. To ensure a closer match of algae dimensions and material properties, future simulations will be run to match parameters which represent experimental data even more closely.

The initial conditions for the simulation were prescribed such that supernatant began at rest and a boundary condition was imposed on the inlet side (left side in FIG. 22B) of 25 mm/s. Once the simulation started, it took some time for the flow field to achieve steady-state. Due to concern with observing cell disruption when the flow was developed, the supernatant was allowed to flow in the domain for 20 ms while the algae were held stationary. After 20 ms, the flow of the supernatant was developed and the algae were allowed to be advected throughout the domain due to drag forces with the supernatant. It should be noted that significant cell disruption may occur as the flow develops due to an unsteady pattern of the flow. Therefore, intermittently starting and stopping the flow may create conditions favorable for cell wall disruption and this phenomenon could be explored in more detail. Images of the supernatant streamlines and algae, as they flow over the obstacle, are displayed in FIG. 22, which shows supernatant streamlines and algae as they move around the obstacle after 60 ms of simulation time. In FIG. 22A, a trimetric view is displayed. In FIG. 22B, a side view is displayed

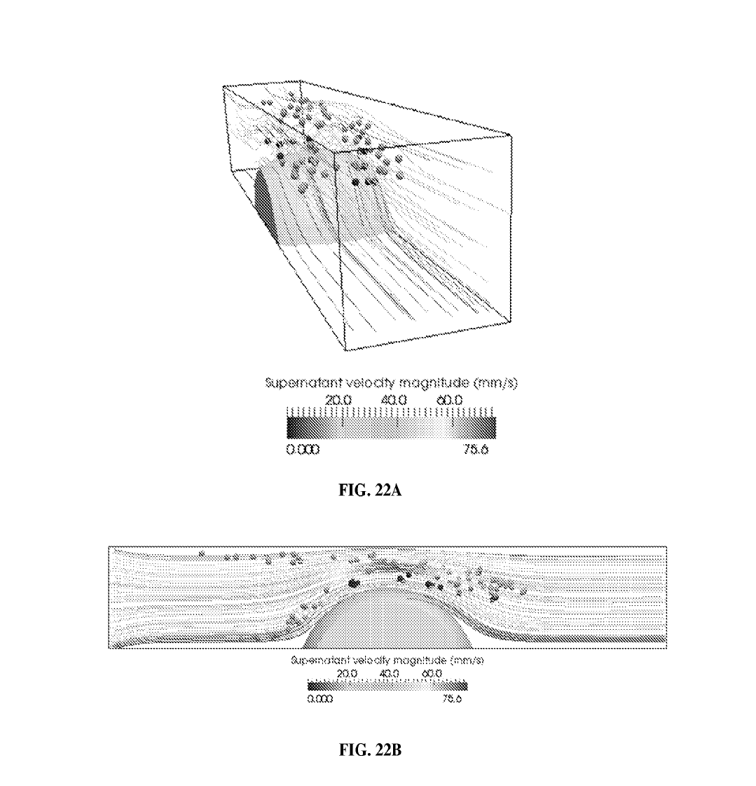

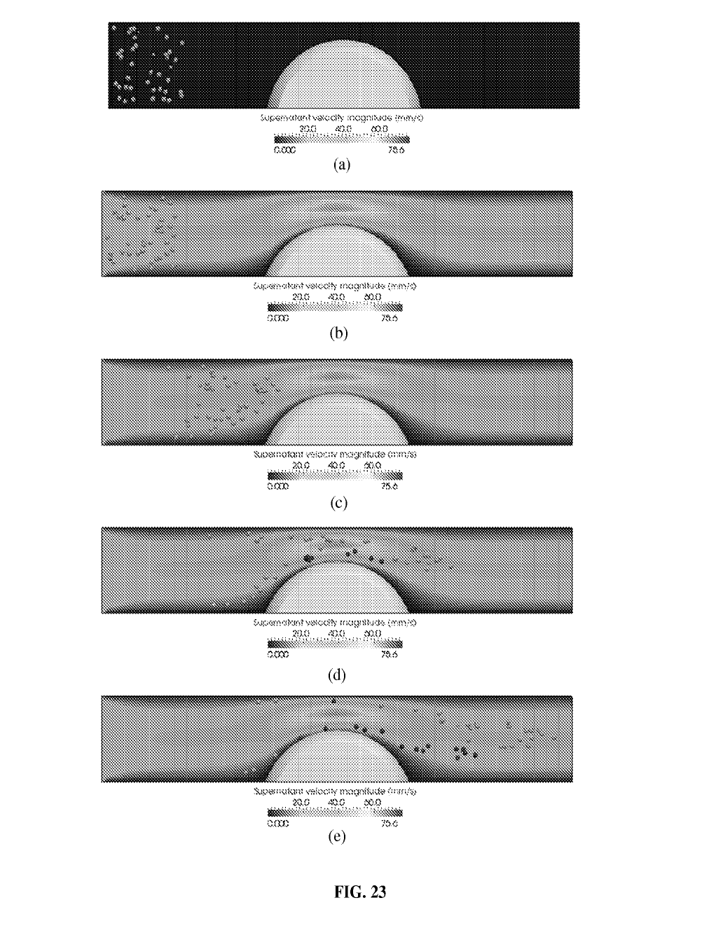

To better illustrate the motion of the algae as they move through the domain, images from the simulation are displayed at various times in FIG. 23. FIG. 23 shows algae positions and supernatent velocity magnitude contour at different times during the simulation. Intact algae are displayed in green, lysed algae are red. Shown are initial conditions (FIG. 23A), after 20 ms (FIG. 23 B), after 40 ms (FIG. 23C), after 60 ms FIG. 23D), and after 80 ms (FIG. 23E).

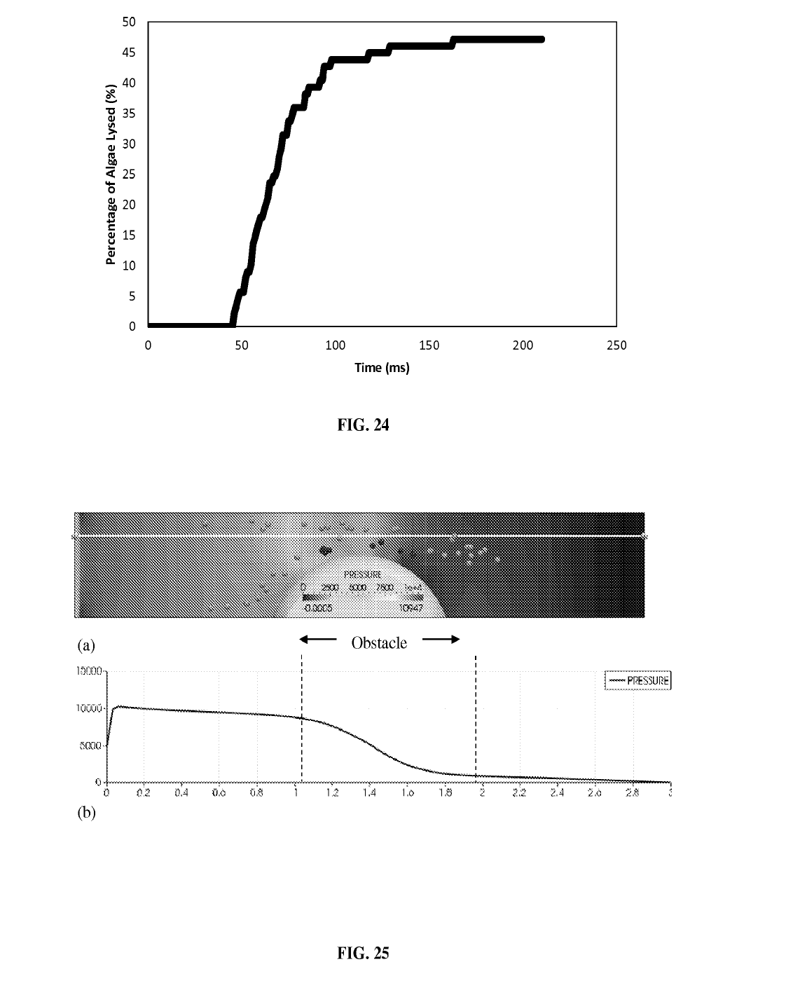

In FIG. 23A, the algae can be seen in their initial position and the supernatant is stationary. In FIG. 6b, the flow in the domain is steady, and the increase in supernatant velocity can be seen as it passes over the obstacle. The supernatant velocity increases to conserve mass as it moves through the constricted region above the obstacle. In the rest of the images of FIG. 23, it can be seen that the algae positions advance in the domain due to drag with the supernatant. Any of the algae which experience a shear stress, calculated from Eq. (5), that exceeds their shear strength, turn from green to red. As the algae pass over the obstacle, only some of the algae are lysed. This can be seen in FIG. 23E where some of the algae that have passed the obstacle are red and some remain green. More detail about when cells are found to be lysed and when they are not is provided in the following sections. From the model, it is possible to ascertain the cumulative percentage of lysed cells vs. time. This data can be seen in FIG. 24.

It can be seen in FIG. 24, that it took approximately 50 ms before any algae were lysed. This is because, the algae were held stationary for 20 ms while the flow developed. Once the algae began to move, it took some time for them to pass into the high strain-rate region above the obstacle. It should also be noted, that not all of the algae were lysed. The simulations were run for about 200 ms which allowed approximately 97% of the algae cells to pass the obstacle. The algae which did not pass the obstacle became trapped in the regions of low supernatant velocity on the surface of the obstacle or on the lower wall immediately before the obstacle.

### Pressure Drop Across the Obstacle

The pressure drop across the obstacle is important in calculating the power consumed to maintain the constant supernatant velocity flow rate Eq. (6). In FIG. 25, the pressure drop across the length of the domain is displayed. This pressure is plotted along the white line in FIG. 25A. The figure shows contour of the pressure in the domain (FIG. 25A) and a graph of the pressure drop across the length of the domain (FIG. 25B)

Beginning at the left side of FIG. 25B, it can be seen that the pressure starts low and then increases to about 1000 g/(s² mm). The reason for this increase at the far left side of the domain is due to the numerical application of the fixed velocity boundary condition. The domain was chosen to be long enough so that this rise in pressure would be negligible when compared to the supernatant's behavior in the rest of the domain. Before and after the obstacle, FIG. 25 displays a linear pressure drop. This is because of the friction with the walls in the domain. The primary pressure drop occurs across the obstacle. Depending on the occlusion ratio, the magnitude of this pressure drop changes. For larger occlusion ratios, the pressure drop is larger. For smaller occlusion ratios, the pressure drop is smaller. This phenomenon agrees with intuition as the larger the occlusion ration, the more resistance the supernatant experiences as it moves through the domain. The increase in resistance in the domain correlates to an increase in the required pumping power (Eq. (6).

### Energy Efficiency of Cell Disruption

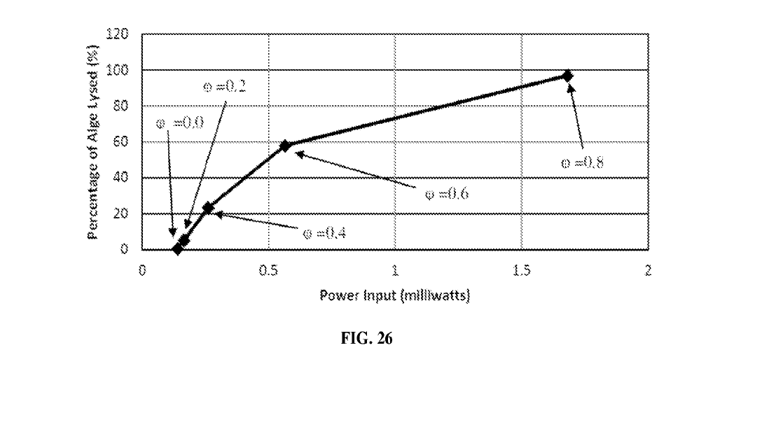

The objective of the current work is to develop a computational framework that can be used to minimize the energy input into the system while lysing the maximum number of cells. To understand how the energy consumption is related to the number of algae cells lysed, a case study was performed in which the occlusion ratio was varied. Accordingly, the energy consumed and the percentage of algae cells lysed was recorded. The results of this study are displayed in FIG. 26, which shows power input vs. percentage of algae lysed.

In FIG. 26, each data point corresponds to a different occlusion ratio. It can be seen that as the occlusion ratio, φ, increases, more power is needed to maintain a constant flow rate. Also, as the occlusion ratio increases, a larger percentage of the algae are lysed. The reason for this is because larger occlusion ratios mean greater strain-rates in the flow. As the occlusion ratio increases, the flow must move faster as it flows around the boundary to conserve mass. The faster flow creates higher strain-rates. Understanding the relationship between the power input and the percentage of algae lysed is important for the practical design of devices which are intended to disrupt cell membranes through fluid-induced shear stresses.

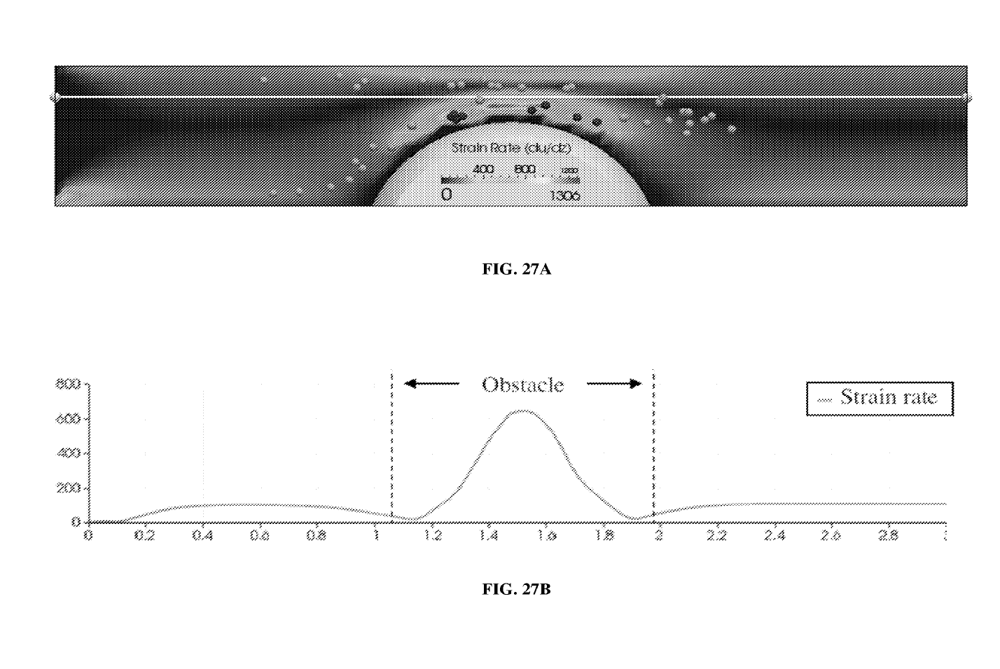

In addition to predicting the percentage of algae lysed, the model described in this work can also be used to understand how the flow configuration affects the strain-rate of the supernatant. Plotting the strain-rate directly can elucidate the locations in the flow where lysing is most likely to occur. An example of this is shown in FIG. 27.

### Investigating the Strain-Rate in the Domain

FIG. 27 shows the contour of the strain-rate of the fluid in the domain (FIG. 27A) and a graph of the strain rate across the length of the domain (FIG. 27B). In FIG. 27B, the strain-rate is plotted along the line shown in FIG. 27A. In FIG. 27B, it can be seen that the strain-rate is low in the beginning of the domain. Once the flow reaches the obstacle, there is a spike in the strain-rate. Once the flow is past the obstacle, the strain-rate decreases. It is of interest to note that although the strain-rate is maximized in the region above the obstacle, the strain-rate in this region varies greatly. The strain-rate is highest near the wall of the domain and the obstacle. However, there is an area in this region where the flow is able to move with little impedance from the walls. This area is denoted with an arrow in FIG. 10a. Algae which pass through this area do not experience a region of high strain-rate and thus are less likely to be lysed. The designers of an experimental device to disrupt the cell walls of algae will want to minimize the presence of such regions.

### CONCLUSION

In this work, a computational framework was developed to predict the lysing of algae cells. A virtual algae shear cell was created to determine the efficiency of lysing. Computational fluid dynamics (CFD) and the discrete element method (DEM) were used to model the supernatant and the algae, respectively. The energy input into the system was calculated by observing the pressure drop across an obstacle placed in the flow to modify the local strain-rates. A correlation was found among the percentage of the domain that was occluded, the energy consumed, and the percentage of algae lysed. Such a framework can be used in the future to optimize the flow configuration such that shear stress from the fluid can be used to lyse algae cells with minimal power input. Along with this framework, experiments would need to be conducted so that mechanical properties, such as the ultimate shear strength of the algae, can be used as a parameter in the model.

The present invention has been described in accordance with several examples, which are intended to be illustrative in all aspects rather than restrictive. Thus, the present invention is capable of many variations in detailed implementation, which may be derived from the description contained herein by a person of ordinary skill in the art.

1. system for producing an algae lysate from an algae culture in an algae culture medium, comprising:
a. a reservoir having a fluid outlet adapted for removal of fluid from the reservoir;
b. a fluid conduit attached to the fluid outlet and comprising one or more lysing structures comprising a fluid flow path and a flow-altering structure that modifies the flow path to generate sufficient force to lyse an algae cell in the algae culture in at least a portion of the flow path at a flow rate generated by either a gravitational head of water of 40 meters or less, 60 psi or less, 30 W or less, or 48,000 Joules (J) or less of energy per Liter of algae culture in algae culture medium.

2. The system of claim 1, comprising algae in algae culture medium within the reservoir at a level above the fluid outlet of the reservoir.

3. The system of claim 1, in which the flow rate is less than or equal to a gravity flow rate.

4. The system of claim 1, in which the flow rate is produced by a pressure of 60 psi or less.

5. The system of claim 1, in which the flow rate is generated by a gravitational head of water of between 8 and 40 meters.

6. The system of claim 1, in which the flow rate is generated by 48,000 J or less of energy per Liter of algae culture in algae culture medium.

7. The system of claim 6, in which the flow rate is generated by from 8 J to 48,000 J of energy per Liter of algae culture in algae culture medium.

8. The system of claim 1, further comprising one or more additional lysing structures in the fluid conduit.

9. The system of claim 1, in which the flow-altering structure is a protuberance within the flow path.

10. The system of claim 1, in which the flow-altering structure is a restriction in a diameter of the flow path.

11. The system of claim 1, in which the flow-altering structure is a venturi.

12. The system of claim 1, in which the flow-altering structure is a bend in the flow path.

13. The system of claim 1, in which the lysing structure at the passive flow rate produces turbulent flow in the lysing structure and walls of the flow path comprise a surface roughness extending into the flow path beyond a turbulent flow viscous sublayer for the medium at the passive flow rate.

14. A method of preparing a product from an algae lysate comprising:
a. growing microalgae in an aqueous medium in a photobioreactor;
b. lysing the microalgae to produce a lysate by flowing the microalgae in the aqueous medium through the lysing structure of claim 1; and
c. producing a product from the lysate.

15. The method of claim 14, wherein the product is a biofuel that is prepared from a hydrophobic fraction of the lysate.

16. The method of claim 14, further comprising prior to lysing the microalgae, pretreating the microalgae to reduce their bursting strength.

17. The method of claim 16, wherein the microalgae are pretreated with either a raised temperature or lowering pH to reduce their bursting strength.

18. A method of lysing algae cells, comprising flowing the microalgae in the aqueous medium through the lysing structure of claim 1.

19. The method of claim 18, further comprising prior to lysing the microalgae, pretreating the microalgae to reduce their bursting strength.

20. The method of claim 19, wherein the microalgae are pretreated with either a raised temperature or lowering pH to reduce their bursting strength.

21. A computer-implemented method of optimizing algae lysis in a lysing structure through which algae is flowed in an aqueous medium, comprising:
a. inputting aqueous medium physical data including temperature, viscosity, density, and solid-fraction, into a fluid dynamics modeling system within a flow domain that geometrically defines a flow path of the aqueous medium through the lysing structure;
b. inputting algae physical data including size, shape, and properties such as bursting strength into a particle modeling system in the flow domain;
c. calculating stresses on the algae in the flow domain for a given aqueous medium flow rate and/or energy input through the flow path; and
d. determining a rate of lysis of cells for the flow rate and/or energy input through the flow path.

22. The method of claim 21, further comprising repeating at least steps c. and d. for a different flow rate and/or energy input and/or flow domain and comparing the results to optimize cell lysis percent and flow rate and/or energy input.

23. A non-transitory computer-readable medium comprising instructions for performing a method according to claim 21 in a processor.
